(19) United States
(12) Patent Application Publication
(10) Pub. No.: US 2008/0219922 A1
(43) Pub. Date: Sep. 11, 2008

(54) Alzheimer's Disease Imaging Agents

(76) Inventors: Mark M. Goodman, Atlanta, GA (US); Fanxing Zeng, Suwanee, GA (US)

Correspondence Address: GREENLEE WINNER AND SULLIVAN P C, 4875 PEARL EAST CIRCLE, SUITE 200, BOULDER, CO, 80301, US

(21) Appl. No.: 12/066,042

(22) PCT Filed: Sep. 11, 2006
(86) PCT No.: PCT/US06/35316

§ 371 Date: Apr. 22, 2008

Related U.S. Application Data
(60) Provisional application No. 60/716,273, filed on Sep. 12, 2005.

(51) Int. Cl. A61K 51/04; C07D 237/26; A61K 31/5025; A61P 25/28; C07D 471/04; A61K 31/437

(52) U.S. Cl. 424/1.65; 544/235; 514/248; 546/121; 514/300

(57) ABSTRACT

This invention provides compounds and methods of imaging amyloid deposits using radiolabeled compounds. This invention also provides a method of inhibiting the aggregation of amyloid proteins to form amyloid plaques or deposits, a method of determining a therapeutic compound's ability to inhibit aggregation of amyloid protein, and a method of delivering a therapeutic agent to amyloid deposits.

### CROSS-REFERENCE TO RELATED APPLICATIONS

This application claims priority to U.S. provisional application Ser. No. 60/716,273, filed Sep. 12, 2005, which application is incorporated by reference to the extent not inconsistent with the disclosure herewith.

### ACKNOWLEDGEMENT OF GOVERNMENT SUPPORT

This invention was made with U.S. Government support under Grant No. R21 MH66622-01 awarded by the National Institute of Health under the National Institute of Mental Health. The U.S. Government has certain rights in this invention.

### BACKGROUND OF THE INVENTION

Alzheimer's disease (AD) is a progressive neurodegenerative disorder characterized by cognitive decline, irreversible memory loss, disorientation, and language impairment. Postmortem examination of AD brain sections reveals abundant amyloid plaques or deposits composed of amyloid-β(Aβ) peptides and numerous neurofibrillary tangles (NFTs) formed by filaments of highly phosphorylated tau proteins. A detailed discussion of this disease can be found in Ginsberg, S. D., et al., “Molecular Pathology of Alzheimer's Disease and Related Disorders,” in Cerebral Cortex. Neurodegenerative and Age-related Changes in Structure and Function of Cerebral Cortex, Kluwer Academic/Plenum, New York (1999), pp. 603-654; Vogelsberg-Ragaglia, V., et al, “Cell Biology of Tau and Cytoskeletal Pathology in Alzheimer's Disease,” Alzheimer's Disease, Lippincot, Williams & Wilkins, Philadelphia, Pa. (1999), pp. 359-372.

The major component of amyloid plaques is a small 39-43 amino acid long β-amyloid peptide that is generated from the cleavage of a larger amyloid precursor protein. However, except for diffuse plaques formed almost exclusively of β-amyloid peptides, amyloid plaques are complex lesions containing numerous associated cellular products. Mutations causing increased production of the 42-43 amino acid form of this peptide have been genetically linked to autosomal dominant familial forms of Alzheimer's disease. Deposits of β-amyloid peptide occur very early in the disease process, long before clinical symptoms develop. Although the exact mechanisms underlying AD are not fully understood, β-amyloids are widely believed to play a causal role in the disease. Whether or not amyloid deposits are causal, they are certainly a key part of the diagnosis. Because amyloid plaques occur early in the disease, the ability to image amyloid plaques would provide a convenient means for early diagnosis and prevention of the disease as well as a method for monitoring effectiveness of therapeutic agents for the disease.

In addition to the role of amyloid deposits in Alzheimer's disease, the presence of amyloid deposits has also been shown in numerous diseases, which highlights the urgent need for efficient imaging agents. Amyloid deposits are shown to be present in diseases such as Mediterranean fever, Muckle-Wells syndrome, idiopathetic myeloma, amyloid polyneuropathy, amyloid cardiomyopathy, systemic senile amyloidosis, amyloid polyneuropathy, hereditary cerebral hemorrhage with amyloidosis, Down's syndrome, Scrapie, Creutzfeldt-Jacob disease, Kuru, Gerstamnn-Straussler-Scheinker syndrome, medullary carcinoma of the thyroid, Isolated atrial amyloid, β₂-microglobulin amyloid in dialysis patients, inclusion body myositis, β₂-amyloid deposits in muscle wasting disease, and Islets of Langerhans diabetes Type II insulinoma.

The direct imaging of amyloid deposits in vivo is difficult, as the deposits have many of the same physical properties (e.g., density and water content) as normal tissues. Attempts to image amyloid deposits using magnetic resonance imaging (MRI) and computer-assisted tomography (CAT) have been disappointing and have detected amyloid deposits only under certain favorable conditions. In addition, efforts to label amyloid deposits with antibodies, serum amyloid P protein, or other probe molecules have provided some selectivity on the periphery of tissues, but have not provided clear imaging of tissue interiors.

There have been various approaches for developing ligands which can specifically and selectively bind amyloid plaques. [Ashburn, T. T., et al., (1996) Chem. Biol. 3:351-358; Zhen, W., et al., (1999) J. Med. Chem. 42:2805-2815]. Examples include highly conjugated chrysamine-G (CG), Congo red (CR), and 3′-bromo- and 3′-iodo derivatives of CG. These compounds have been shown to bind selectively to amyloid beta peptide aggregates in vitro as well as to fibrillar amyloid beta deposits in AD brain sections [Mathis, C. A., et al., Proc. XIIth Intl. Symp. Radiopharm. Chem., Uppsala, Sweden:94-95 (1997)]. However, ligands useful for detecting amyloid plaque aggregates in the living brain must cross the intact blood-brain barrier. Thus, ligands that are relatively small in size and lipophilic have been sought as candidate imaging agents for amyloid plaques.

The development of radiolabeled ligands to image amyloid deposits using positron emission tomography (PET) and single photon emission computed tomography (SPECT) has been underway for some time. Highly conjugated thioflavins (S and T) are commonly used as dyes for staining the amyloid beta aggregates in the AD brain [Elhaddaoui, A., et al., Biospectroscopy 1: 351-356 (1995)]. These compounds are based on benzothiazole, which is relatively small in molecular size. However, thioflavins contain an ionic quarternary amine, which is permanently charged and unfavorable for brain uptake.

There have been further efforts in developing ligands for imaging amyloid deposits. U.S. Pat. No. 6,696,039, US 2004/0131545, and WO 02/085903 disclose thioflavin derivatives as amyloid plaque aggregation inhibitors and diagnostic imaging agents; U.S. Pat. No. 6,001,331 discloses a method of imaging amyloid deposits using radiolabeled benzothiazole derivatives; WO 2004/032975 describes various biphenyls and fluorenes as imaging agents in Alzheimer's disease; WO 2004/064869 discloses metal-chelating agents for the diagnosis, prevention, and treatment of pathophysiological conditions associated with amyloid accumulation; US 2005/0043377 describes further thioflavin derivatives for in vivo imaging and prevention of amyloid deposition.

Considering that Alzheimer's disease affects approximately 20 to 40% of the population over 80 years of age, the fastest growing age group in the United States and other post-industrial countries, there is a continuing need for an agent with high selectivity and specificity for binding amyloid deposits or plaques which can be used in a simple, noninvasive method for in vivo imaging and quantitating amyloid deposits in a patient. To this end, the present invention provides novel compounds and methods for imaging amyloid deposits and inhibiting formation of amyloid deposits using such compounds.

### SUMMARY OF THE INVENTION

The present invention provides novel compounds of Formula I, II or III that are useful for detecting and quantitating amyloid deposits. The compounds are also useful in inhibiting the aggregation of amyloid proteins to form amyloid deposits and in delivering a therapeutic agent selectively and specifically to amyloid deposits.

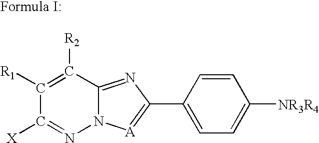

wherein R₁ and R₂ are each independently selected from the group consisting of: H, CH₃, CH₂(CH₂)ₙCH₃, CH₂(CH₂)ₙCH₂F, CH₂CH═CH(CH₂)ₙF, (CH₂)ₙCH═CH(CH₂)ₙI, OH, OCH₃, OCH₂(CH₂)ₙCH₃, OCH₂(CH₂)ₙCH₂F, OCH₂CH═CH(CH₂)ₙF, O(CH₂)ₙCH═CH(CH₂)ₙI, SH, SCH₃, SCH₂(CH₂)ₙCH₃, SCH₂(CH₂)ₙCH₂F, SCH₂CH═CH(CH₂)ₙF, S(CH₂)ₙCH═CH(CH₂)ₙI (n=0-7); aromatic, haloaromatic, heteroaromatic, haloheteroaromatic, cyclic, halogen, and halocyclic; X is selected from the group consisting of F, Cl, Br, I, CH₂(CH₂)ₙCH₂F, CH₂CH═CH(CH₂)ₙF, (CH₂)ₙCH═CH(CH₂)ₙI, OH, OCH₃, OCH₂(CH₂)ₙCH₃, OCH₂(CH₂)ₙCH₂F, OCH₂CH═CH(CH₂)ₙF, O(CH₂)ₙCH═CH(CH₂)ₙI, SH, SCH₃, SCH₂(CH₂)ₙCH₃, SCH₂(CH₂)ₙCH₂F, SCH₂CH═CH(CH₂)ₙF, S(CH₂)ₙCH═CH(CH₂)ₙI (n=0-7), and Tc-99m complexed compounds; A is CH or N; R₃ and R₄ are each independently selected from the group consisting of: hydrogen, C₁₋₄ alkyl, C₂₋₄ aminoalkyl, C₁₋₄ haloalkyl, and haloarylalkyl, or R₃ and R₄ are taken together with the nitrogen to which they are attached to form a 5- to 7-member heterocyclic ring optionally having O, S or NR₅ in said ring, where R₅ is hydrogen or C₁₋₄ alkyl, provided that both R₁ and R₂ are not both H when A is CH, and X is Br, I, F, ¹²⁵I, ¹³¹I, ¹²³I, ¹⁸F, ⁷⁶Br, ⁷⁷Br, haloalkyl, Sn(alkyl)₃ or -L-Ch, where L is —(CH₂)ₙ— or —(CH₂)ₙ—C(O)—, where n is 0 to 5 and Ch is a tetradentate ligand capable of complexing with a metal.

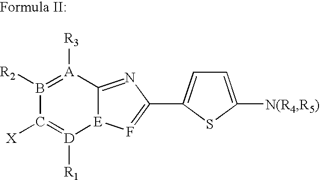

wherein R₁, R₂, R₃ are each independently selected from the group consisting of: H, CH₃, CH₂ (CH₂)ₙCH₃, CH₂(CH₂)ₙCH₂F, CH₂CH═CH(CH₂)ₙF, (CH₂)ₙCH═CH(CH₂)ₙI, OH, OCH₃, OCH₂(CH₂)ₙCH₃, OCH₂(CH₂)ₙCH₂F, OCH₂CH═CH(CH₂)ₙF, O(CH₂)ₙCH═CH(CH₂)ₙI, SH, SCH₃, SCH₂(CH₂)ₙCH₃, SCH₂(CH₂)ₙCH₂F, SCH₂CH═CH(CH₂)ₙF, S(CH₂)ₙCH═CH(CH₂)ₙI (n=0-7), aromatic, haloaromatic, heteroaromatic, haloheteroaromatic, cyclic, halogen and halocyclic; R₄, R₅ are each independently selected from the group consisting of: H, CH₃, CH₂ (CH₂)ₙCH₃, CH₂(CH₂)ₙCH₂F, CH₂CH═CH(CH₂)ₙF, (CH₂)ₙCH═CH(CH₂)ₙI, OH, OCH₃, OCH₂(CH₂)ₙCH₃, OCH₂(CH₂)ₙCH₂F, OCH₂CH═CH(CH₂)ₙF, O(CH₂)ₙCH═CH(CH₂)ₙI, SH, SCH₃, SCH₂(CH₂)ₙCH₃, SCH₂(CH₂)ₙCH₂F, SCH₂CH═CH(CH₂)ₙF, S(CH₂)ₙCH═CH(CH₂)ₙI (n=0-7), aromatic, haloaromatic, heteroaromatic, haloheteroaromatic, cyclic, and halocyclic, or R₄ and R₅ are taken together with the nitrogen to which they are attached to form a 5- to 7-member heterocyclic ring optionally having O, S or NR₆ in said ring, where R₆ is hydrogen or C₁₋₄ alkyl; X is selected from the group consisting of: F, Cl, Br, I, CH₂(CH₂)ₙCH₂F, CH₂CH═CH(CH₂)ₙF, (CH₂)ₙCH═CH(CH₂)ₙI, OH, OCH₃, OCH₂(CH₂)ₙCH₃, OCH₂(CH₂)ₙCH₂F, OCH₂CH═CH(CH₂)ₙF, O(CH₂)ₙCH═CH(CH₂)ₙI, SH, SCH₃, SCH₂(CH₂)ₙCH₃, SCH₂(CH₂)ₙCH₂F, SCH₂CH═CH(CH₂)ₙF, S(CH₂)ₙCH═CH(CH₂)ₙI (n=0-7) and Tc-99m complexed compounds; A, B, C, D are each independently selected from the group consisting of: C, CH, N, and N⁺O⁻; E, F are each independently selected from the group consisting of: C, CH and N.

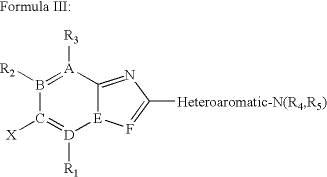

wherein R₁, R₂, R₃ are each independently selected from the group consisting of: H, CH₃, CH₂(CH₂)ₙCH₃, CH₂(CH₂)ₙCH₂F, CH₂CH═CH(CH₂)ₙF, (CH₂)ₙCH═CH(CH₂)ₙI, OH, OCH₃, OCH₂(CH₂)ₙCH₃, OCH₂(CH₂)ₙCH₂F, OCH₂CH═CH(CH₂)ₙF, O(CH₂)ₙCH═CH(CH₂)ₙI, SH, SCH₃, SCH₂(CH₂)ₙCH₃, SCH₂(CH₂)ₙCH₂F, SCH₂CH═CH(CH₂)ₙF, S(CH₂)ₙCH═CH(CH₂)ₙI (n=0-7), aromatic, haloaromatic, heteroaromatic, haloheteroaromatic, cyclic, halogen and halocyclic; R₄, R₅ are each independently selected from the group consisting of: H, CH₃, CH₂(CH₂)ₙCH₃, CH₂(CH₂)ₙCH₂F, CH₂CH═CH(CH₂)ₙF, (CH₂)ₙCH═CH(CH₂)ₙI, OH, OCH₃, OCH₂(CH₂)ₙCH₃, OCH₂(CH₂)ₙCH₂F, OCH₂CH═CH(CH₂)ₙF, O(CH₂)ₙCH═CH(CH₂)ₙI, SH, SCH₃, SCH₂(CH₂)ₙCH₃, SCH₂(CH₂)ₙCH₂F, SCH₂CH═CH(CH₂)ₙF, S(CH₂)ₙCH═CH(CH₂)_{nxI (n=}0-7), aromatic, haloaromatic, heteroaromatic, haloheteroaromatic, cyclic, and halocyclic, or R₄ and R₅ are taken together with the nitrogen to which they are attached to form a 5- to 7-member heterocyclic ring optionally having O, S or NR₆ in said ring, where R₆ is hydrogen or C₁₋₄ alkyl; X is selected from the group consisting of: F, Cl, Br, I, CH₂(CH₂)ₙCH₂F, CH₂CH═CH(CH₂)ₙF, (CH₂)ₙCH═CH(CH₂)ₙI, OH, OCH₃, OCH₂(CH₂)ₙCH₃, OCH₂(CH₂)ₙCH₂F, OCH₂CH═CH(CH₂)ₙF, O(CH₂)ₙCH═CH(CH₂)ₙI, SH, SCH₃, SCH₂(CH₂)ₙCH₃, SCH₂(CH₂)ₙCH₂F, SCH₂CH═CH(CH₂)ₙF, S(CH₂)ₙCH═CH(CH₂)ₙI (n=0-7), and Tc-99m complexed compounds; A, B, C, D are each independently selected from the group consisting of: C, CH, N, and N⁺O⁻; E, F are each independently selected from the group consisting of: C, CH and N.

In a particular example, R₁ and R₂ are not both H in Formula I. In a particular example, R₂ and R₃ are not both H in Formula II or Formula III. In a particular example, R₃ and R₄ are both CH₃ in Formula I. In a particular example, R₄ and R₅ are both CH₃ in Formula II and III. In a particular example, one of R₂ and R₃ are H and the other of R₂ and R₃ are CH₃ in Formula II or III. In a particular example, in Formula II or III, A, B, and C are C; F is CH; and D and E are N. Preferred compounds of the invention include compounds of Formula I wherein A is CH or N, either R₁ or R₂ is methyl, and R₃ and R₄ are methyl. Particularly preferred compounds of Formula I are those wherein either R₁ or R₂ is methyl, R₃ and R₄ are methyl, and A is CH, X is F, ¹⁸F, I, ¹²³I or ¹²⁴I. In a particular example, X is F, ¹⁸F, I, ¹²³I or ¹²⁴I in a compound of Formula I, II or III. In a particular example, X is F or ¹⁸F in a compound of Formula I, II or III. In a particular example, X is, I, ¹²³I or ¹²⁴I in a compound of Formula I, II or III.

In a particular example, one of R₁ and R₂ are H and the other of R₁ and R₂ are CH₂CH₂F in Formula I. In a particular example, R₁, R₂ and R₃ are all H in Formula II or Formula III. In a particular example, one of R₂ and R₃ are H and the other of R₂ and R₃ are CH₂CH₂F in Formula II or II. In a particular example, one of X, R₁ and R₂ is halogenated in Formula I. In a particular example, one of X, R₁ and R₂ is H in Formula I. In a particular example, E is N in Formula II. In a particular example, E and one of A, B, C, D are N in Formula II. In a particular example, E and one or more of A, B, C, D are N in Formula II. In a particular example, X is a halogen or halogen-containing substituent in Formula II. In a particular example, R2 is a halogen or halogen-containing substituent in Formula II. In a particular example, E is N in Formula II. In a particular example, E and one of C, D, A are N in Formula III. In a particular example, E and one or more of A, C, D are N in Formula III. In a particular example, the heteroaromatic ring in Formula III is halogenated. In a particular example, X and R2 are halogen or halogenated in Formula III. In a particular example, A and D are N and the heteroaromatic ring is halogenated in Formula III. In a particular example, the heteroaromatic ring in Formula III is pyridine or pyrimidine and may or may not be halogenated, particularly with I or F.

Any of F, Cl, Br, I or C in the formulas shown above may be in stable isotopic or radioisotopic form. Particularly useful radioisotopic labels are ¹⁸F, ¹²³I, ¹²⁵I, ¹³¹I, ⁷⁶Br, ⁷⁷Br and ¹¹C. Compounds of the invention bind to amyloid deposits with high affinity and selectivity. The inventive compounds labeled with an appropriate radioisotope are useful as imaging agents for visualizing the location and density of amyloid deposits by PET and SPECT imaging. Accordingly, the labeled compounds of the invention are useful for diagnostic imaging and evaluating efficacy of any therapeutic compounds for Alzheimer's disease. The method of imaging amyloid deposits provided comprises (a) introducing into a subject a detectable quantity of a labeled compound of Formula I, II or III, and/or a pharmaceutically acceptable salt, ester or amide thereof; (b) allowing sufficient time for the labeled compound to become associated with amyloid deposits; and (c) detecting the labeled compound associated with one or more amyloid deposits.

The present invention also provides diagnostic compositions comprising a radiolabeled compound of Formula I, II or III, and/or a pharmaceutically acceptable carrier or diluent. Also within the scope of the invention are pharmaceutical compositions which comprise a compound of Formula I, II or III, and/or a pharmaceutically acceptable carrier or diluent. The pharmaceutical compositions are useful for inhibiting the aggregation of amyloid proteins or for delivering a therapeutic agent in a subject. Also provided are pharmaceutically acceptable salts of the compounds of Formula I, II, or III. Also provided herein are methods of making the compounds of Formula I, II, or III. Methods of quantitating amyloid deposits are also provided herein.

### BRIEF DESCRIPTION OF THE FIGURES

FIG. 1 shows the time-activity curves of brain regions for [¹⁸F]-FZ 202-1.

FIG. 2 shows the time-activity curves of brain regions for [¹⁸F] FZ 202-2.

FIG. 3 shows in vitro autoradiographic detection of Aβ amyloid deposits with [¹⁸F] FZ 202-1 in postmortem brain tissue sections of frontal lobe from an AD patient. A: AD tissue+[¹⁸F] FZ 202-1. B: [¹⁸F] FZ 202-1+1 mM PIB. C: [¹⁸F] FZ 202-1+10 mM IMPY.

### DETAILED DESCRIPTION OF THE INVENTION

In general, the terms and phrases used herein have their art-recognized meaning, which can be found by reference to standard texts, journal references and contexts known to those skilled in the art. The definitions provided are intended to clarify their specific use in the context of the invention.

The term “pharmaceutically acceptable salt” as used herein refers to those carboxylate salts or acid addition salts of the compounds of the present invention which are suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention. The term “salts” refers to the relatively nontoxic, inorganic and organic acid addition salts of compounds of the present invention. Also included are those salts derived from non-toxic organic acids such as aliphatic mono and dicarboxylic acids, for example acetic acid, phenyl-substituted alkanoic acids, hydroxy alkanoic and alkanedioic acids, aromatic acids, and aliphatic and aromatic sulfonic acids. These salts can be prepared in situ during the final isolation and purification of the compounds or by separately reacting the purified compound in its free base form with a suitable organic or inorganic acid and isolating the salt thus formed. Further representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactiobionate and laurylsulphonate salts, propionate, pivalate, cyclamate, isethionate, and the like. These may include cations based on the alkali and alkaline earth metals, such as sodium, lithium, potassium, calcium, magnesium, and the like, as well as nontoxic ammonium, quaternary ammonium and amine cations including, but not limited to ammonium, tetramethylammonium, tetraethylammonium, methylamine, dimethylamine, trimethylamine, triethylamine, ethylamine, and the like. See, for example, Berge S. M, et al., Pharmaceutical Salts, J. Pharm. Sci. 66:1-19 (1977) which is incorporated herein by reference.

Similarly, the term, “pharmaceutically acceptable carrier,” as used herein, is an organic or inorganic composition which serves as a carrier/stabilizer/diluent of the active ingredient of the present invention in a pharmaceutical or diagnostic composition. In certain cases, the pharmaceutically acceptable carriers are salts. Further examples of pharmaceutically acceptable carriers include but are not limited to water, phosphate-buffered saline, saline, pH controlling agents (e.g. acids, bases, buffers), stabilizers such as ascorbic acid, isotonizing agents (e.g. sodium chloride), aqueous solvents, a detergent (ionic and non-ionic) such as polysorbate or TWEEN 80.

The term “alkyl” as used herein by itself or as part of another group refers to both straight and branched chain radicals of up to 8 carbons, preferably 6 carbons, more preferably 4 carbons, such as methyl, ethyl, propyl, isopropyl, butyl, t-butyl, and isobutyl.

The term “alkoxy” is used herein to mean a straight or branched chain alkyl radical, as defined above, unless the chain length is limited thereto, bonded to an oxygen atom, including, but not limited to, methoxy, ethoxy, n-propoxy, isopropoxy, and the like. Preferably the alkoxy chain is 1 to 6 carbon atoms in length, more preferably 1-4 carbon atoms in length.

The term “monoalkylamine” as used herein by itself or as part of another group refers to an amino group which is substituted with one alkyl group as defined above.

The term “dialkylamine” as employed herein by itself or as part of another group refers to an amino group which is substituted with two alkyl groups as defined above.

The term “halo” employed herein by itself or as part of another group refers to chlorine, bromine, fluorine or iodine.

The term “aryl” as employed herein by itself or as part of another group refers to monocyclic or bicyclic aromatic groups containing from 6 to 12 carbons in the ring portion, preferably 6-10 carbons in the ring portion, such as phenyl, naphthyl or tetrahydronaphthyl.

The term “heterocycle” or “heterocyclic ring”, as used herein except where noted, represents a stable 5- to 7-membered mono-heterocyclic ring system which may be saturated or unsaturated, and which consists of carbon atoms and from one to three heteroatoms selected from the group consisting of N, O, and S, and wherein the nitrogen and sulfur heteroatom may optionally be oxidized. Especially useful are rings contain one nitrogen combined with one oxygen or sulfur, or two nitrogen heteroatoms. Examples of such heterocyclic groups include piperidinyl, pyrrolyl, pyrrolidinyl, imidazolyl, imidazlinyl, imidazolidinyl, pyridyl, pyrazinyl, pyrimidinyl, oxazolyl, oxazolidinyl, isoxazolyl, isoxazolidinyl, thiazolyl, thiazolidinyl, isothiazolyl, homopiperidinyl, homopiperazinyl, pyridazinyl, pyrazolyl, and pyrazolidinyl, most preferably thiamorpholinyl, piperazinyl, and morpholinyl.

The term “heteroatom” is used herein to mean an oxygen atom (“O”), a sulfur atom (“S”) or a nitrogen atom (“N”). It will be recognized that when the heteroatom is nitrogen, it may form an NR^{a}R^{b} moiety, wherein R^{a} and R^{b} are, independently from one another, hydrogen or C₁₋₄ alkyl, C₂₋₄ aminoalkyl, C₁₋₄ halo alkyl, halo benzyl, or R¹ and R² are taken together to form a 5- to 7-member heterocyclic ring optionally having O, S or NRC in said ring, where R^{c} is hydrogen or C₁₋₄ alkyl.

The term “heteroaromatic” is used herein to mean an aromatic ring substituted with one or more heteroatoms and may contain substituents including halogens, alkyl, alkoxy, alkylhio, alkenyl, allynyl, haloalkyl, haloalkoxy, haloalkylhio, haloalkenyl, haloalkynyl, haloaromatic and haloheteroaromatic.

In order to develop new imaging agents for amyloid plaques or deposits, the inventors herein identified a new class of thioflavin derivatives, 2-(4′-dimethylamino-)phenyl-imidazodiazines which have high affinity for amyloid-β (Aβ) plaques and which can be readily labeled with positron emitting radioelements or single photon emitting radio-elements attached directly to or via a linker molecule to the diazine ring. The unique aspect of one group of the provided compounds is the introduction of a pyridazine ring which contains fluorine or iodine on the 6-position and methyl groups on either the 7- or 8-position, into the thioflavin motif, which gives rise to high affinity to amyloid-β (Aβ) plaques and allows introduction of radiohalogens by heteroaromatic nucleophilic displacement. This is especially important in the case of fluorine-18. Fluorine-18 is the most desirable positron emitting radioelement for labeling amyloid-β (Aβ) plaque imaging agents because its 110 minute half-life allows sufficient time (3×110 minutes) for radiosynthesis and for purification of the final product suitable for subsequent human administration. Secondly, fluorine-18 can be prepared in curie quantities as fluoride ion which can be used for automated radiosynthetic procedures, as has been developed for [¹⁸F]FACBC [McConathy, et al., Applied Radiation and Isotopes 58:657 (2003)]. Radiopharmaceuticals of very high specific activity can be obtained in a theoretical, 1.7 Ci/nmol specific activity that can be calculated for a no-carrier-added fluorine-18 fluoride ion nucleophilic substitution reaction. Fluorine-18 is also the lowest energy positron emitter (0.635 MeV, 2.4 mm positron range) which affords the highest spatial resolution in PET images. Finally, the 110-minute half-life allows sufficient time for central manufacturing site and for regional distribution of the amyloid-β (Aβ) plaque imaging agent to hospitals without on site particle accelerators; a regional distribution radius of 200 mile is feasible as has been shown with [¹⁸F]FDG.

The compounds of the invention are represented by Formulas I, II and III as shown herein. It is noted that all compounds depicted in Formulas I, II and III are intended to be disclosed to the same extent as if they were specifically shown in this disclosure. It is intended that all individual compounds separately and all possible groupings of compounds of Formulas I, II and III can be included and/or excluded in the claims. In addition, all possibilities for each variable are intended to be disclosed to the same extent as if they were specifically shown in this disclosure. It is intended that all individual members of all groups and all possible groups provided herein can be included and/or excluded in the claims.

Specific compounds having formula I include the following:

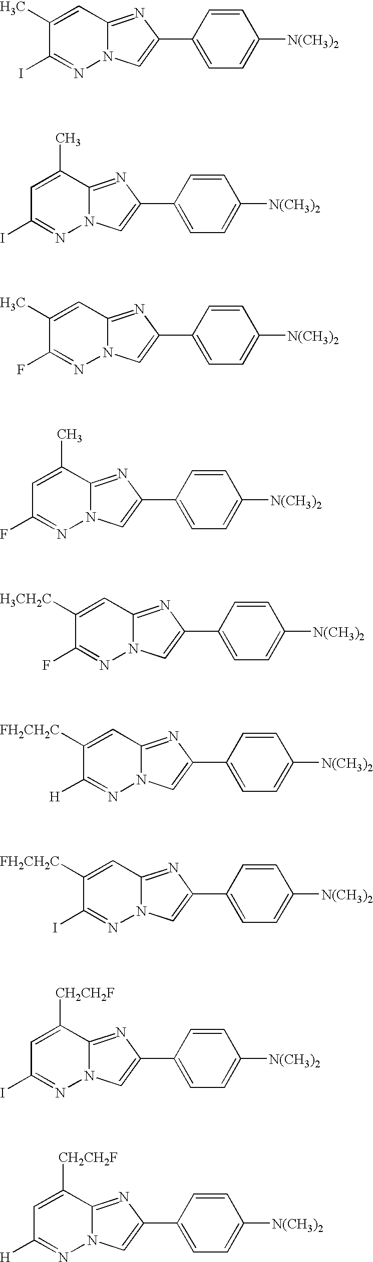

Specific compounds of Formula II include the following:

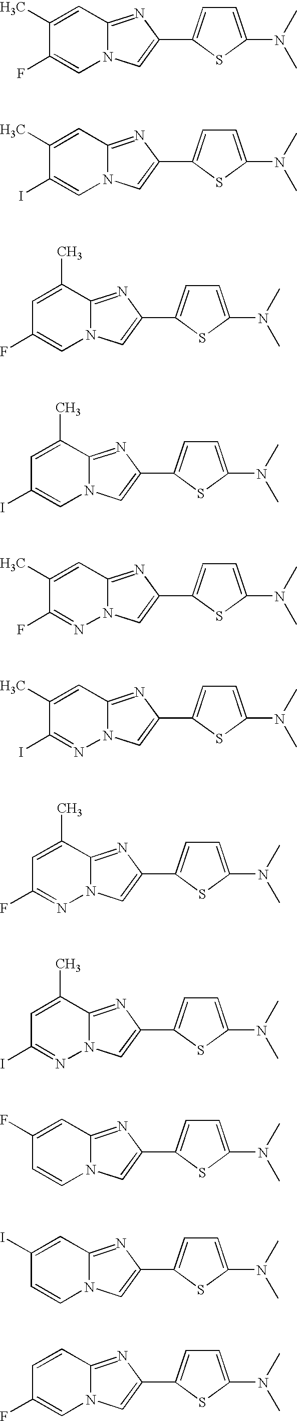

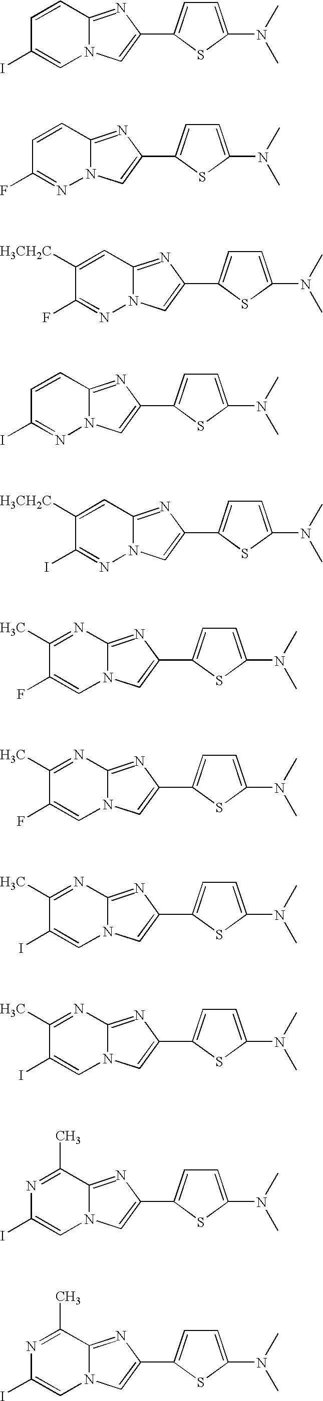

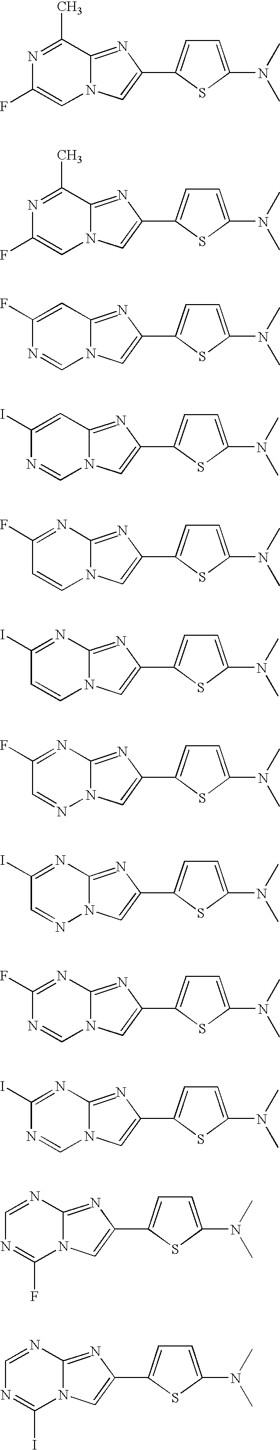

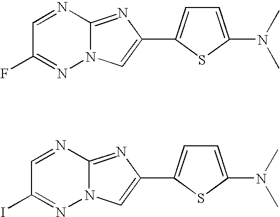

Specific compounds of Formula III include the following:

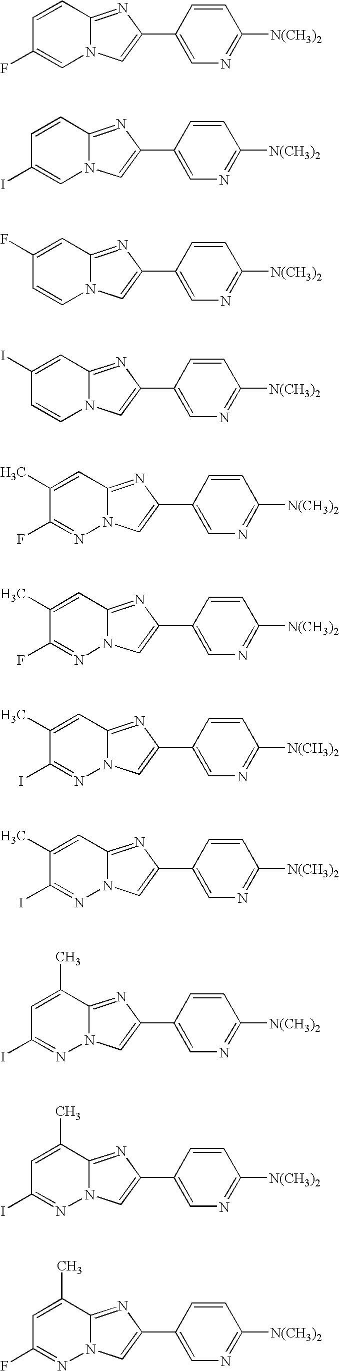

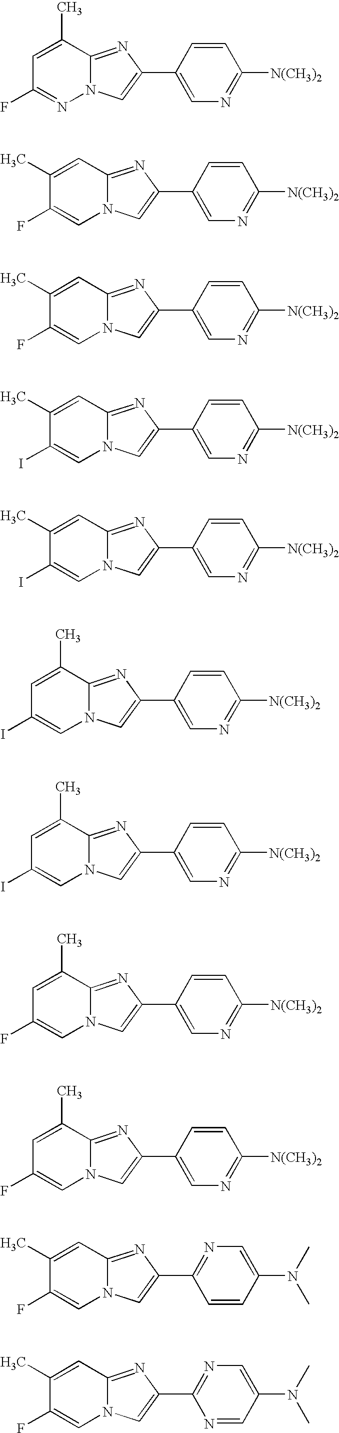

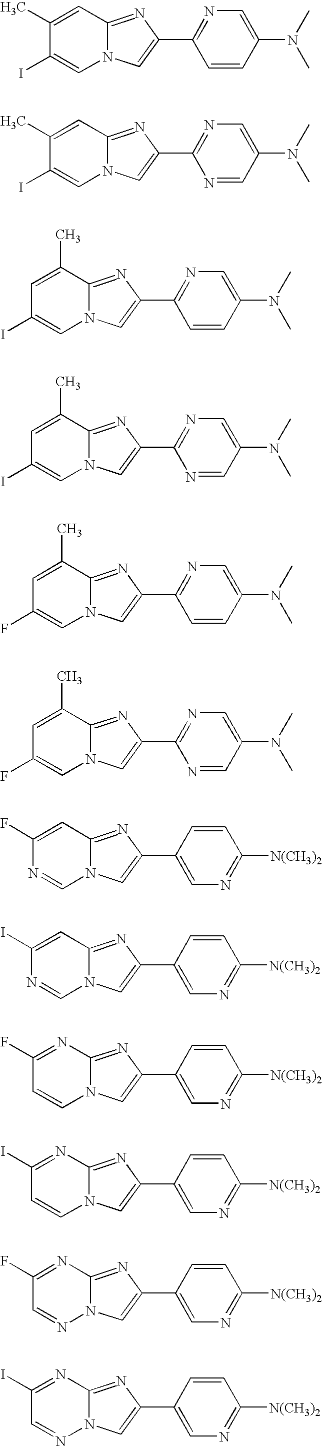

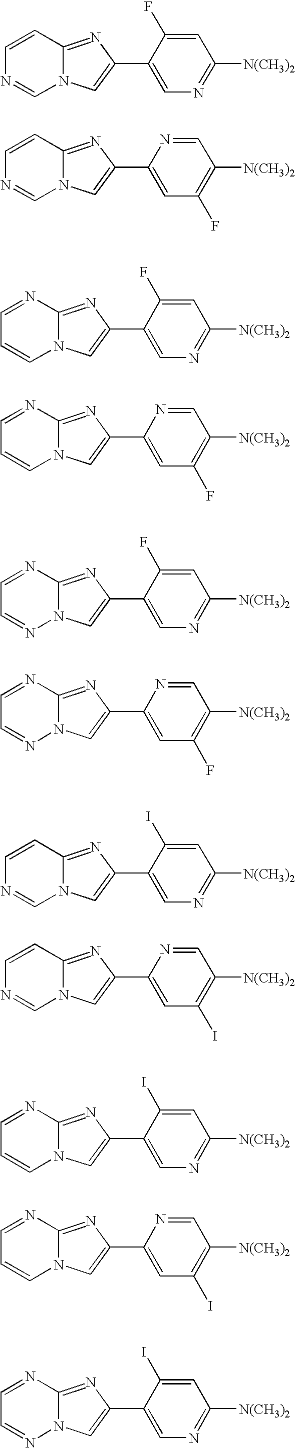

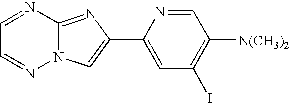

Examples of the compounds represented by Formula I have been evaluated for their binding affinities via the binding competition with IMPY using human AD cortical tissues. The inhibition constants Ki (nM), for the competitive inhibition of the new unlabeled 2-(4′-dimethylamino-)phenyl-imidazo[1,2-b]pyridazines for amyloid-β (Aβ) plaques and neurofibrillary tangles vs [¹²⁵I]MPY is shown in Table 1. These results demonstrate that certain compounds of the invention have higher affinity for amyloid plaques than IMPY. The rank in affinity of the 2-(4′-dimethylamino-)phenyl-imidazodiazine analogues were FZ 202-1>PIB>IMPY>FZ 201-1>>FZ 201-2>FZ 202-2.

TABLE 1
Inhibition constants

| | Ki (nM) vs |
|---|---|
| Compound | [¹²⁵I]- IMPY |
| 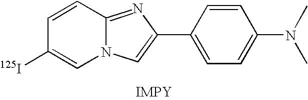 | 10.4 |
| 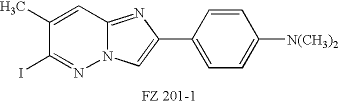 | 20 |
| 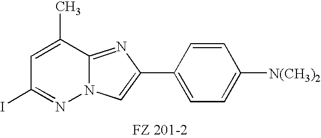 | 91.6 |
| 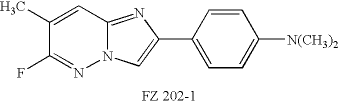 | 2.7 |
| 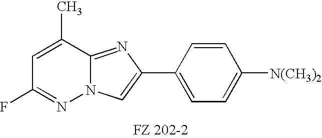 | 131.8 |
| 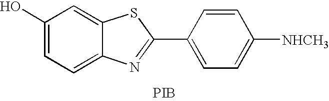 | 7.8 |

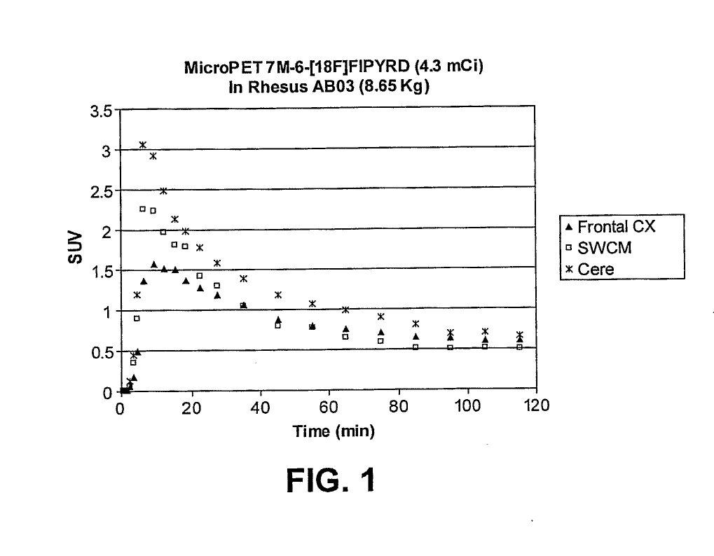

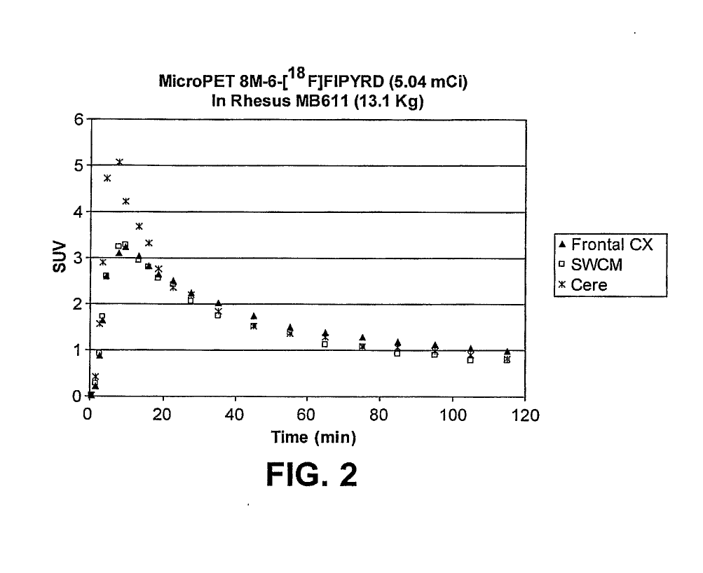

In order for a compound to be an ideal imaging agent for amyloid plaques, it should exhibit certain physicochemical characteristics. For example, the compound should have a Log P_{7.4} value of 2.0-4.0 and good early peak brain penetrance of less than 10 min, and show rapid washout from all brain regions i.e. cerebellum, cortex and subcortical white matter. As shown in Table 2, two of the inventive compounds (FZ 202-1 and FZ 202-O₂) have Log P_{7.4} values well within the desirable range, 2.69 and 2.83, respectively, which are lower than that observed with the known compound, IMPY. In order to assess brain uptake and clearance, FZ 202-1 and FZ 202-2 were radiolabeled with fluorine-18. The kinetics of fluorine-18 labeled FZ 202-1 and FZ 202-2 in brain were determined by microPET in rhesus monkeys. Since these animals are normal i.e., have no amyloid deposits in their brains, this experiment should reflect brain entry and clearance from normal brain tissue. The time-activity curves of [¹⁸F] FZ 202-1 and [¹⁸F] FZ 202-2 are shown in FIGS. 1 and 2, respectively. These studies demonstrate that both compounds penetrate the blood-brain barrier easily after intravenous injection, with maximum brain radioactivity concentration (SUV) of 1.6-3.1 at 9 min for [¹⁸F]-FZ 202-1 and 3.3-5.1 at 9 min for [¹⁸F]-FZ 202-2. Relatively fast nonspecific binding clearance was observed for both compounds, with the radioactivity ratios of peak-to-115 min in cerebellum, frontal cortex and subcortical white matter devoid of specific binding sites between 2.5-4.9 for [¹⁸F]-FZ 202-1 and 3.2-6.0 for [¹⁸F]-FZ 202-2. No bone uptake of radioactivity was observed in the skull after the intravenous administration of [¹⁸F]-FZ 202-1 and [¹⁸F]-FZ 202-2 to the monkeys. The results of competition binding assay and in vivo biodistribution study demonstrate that [¹⁸F]-FZ 202-1 is an excellent imaging agent for amyloid deposits using PET.

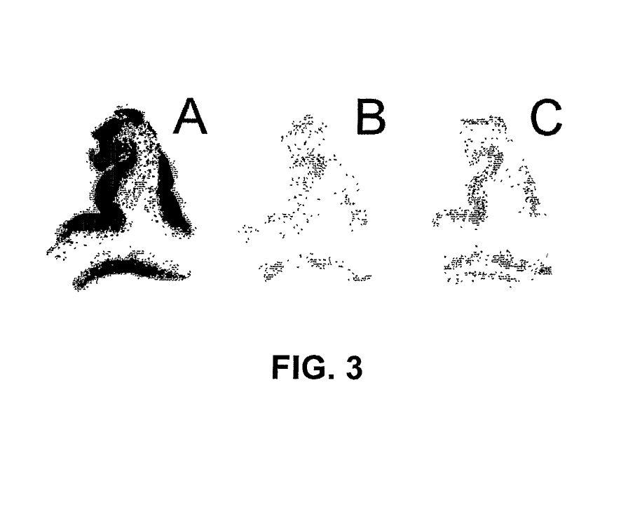

TABLE 2

| Compound | Log P_{7.4} |
|---|---|
| FZ 202-1 | 2.69 |
| FZ 202-2 | 2.83 |
| IMPY | 3.58 |

[¹⁸F] FZ 202-1 plaque labeling was evaluated by in vitro film autoradiography as shown in FIG. 3. Specific binding of [¹⁸F] FZ 202-1 to amyloid plaques in sections from postmortem AD brains was clearly observed in cortical gray matter, but not in the white matter, and the specific binding was eliminated in the AD specimen with the pretreatment with nonradioactive PIB and IMPY.

The studies described above indicate that the compounds of Formula I are excellent imaging agents for amyloid plaques. The inventive compounds can readily penetrate the intact blood-brain barrier and be retained in the brain sufficiently long enough for imaging. These compounds exhibit specific brain uptake over other tissues in vivo. Compounds of formulas II and III exhibit physicochemical characteristics similar to those observed with the compounds of formula I (i.e., penetration across the blood-brain barrier and selective and specific binding to amyloid plaques as well as the selective brain uptake) and thus can be used as imaging agents for amyloid plaques. Those skilled in the art can synthesize any compound of formula I, II or III according to the description provided herein, combined with the knowledge readily available in the art without undue experimentation. The skilled artisan can also evaluate a compound of the invention as an imaging agent for amyloid plaques by various art-known methods and assays as disclosed in the present application. For example, a given compound can be tested for binding specificity and selectivity for amyloid deposits in an in vitro competitive binding assay using suitable cells, tissues or beta amyloid peptides, along with a known imaging agent as a control, as described in the present application. If the compound shows desired binding characteristics for amyloid plaques, it can then be further evaluated in vivo, i.e., for brain uptake, selective and specific binding for amyloid deposits, by measuring distribution in various tissues after administration into an animal (e.g. rhesus monkey).

The compounds of the above Formulas I, II, and III can be prepared by reactions described in the following Schemes.

Schemes 1 through 6 depict synthetic routes for preparing 7-substituted phenyl-imidazo[1,2-a]pyridine derivatives of the present invention. The initial formation of 7-substituted phenyl-imidazo[1,2-a]pyridine, 1, was readily accomplished by condensation reaction between commercially available 2-amino-4-methyl-5-bromopyridine and 2-bromo-4′-dimethylaminoacetophenone [Diwu, Z.; Beachdel, C.; and Klaubert, D. H. Tetrahedron Lett., 39:4987-4990 (1998)] in the presence of a mild base such as sodium bicarbonate. Palladium catalyzed coupling of 1 with tributyl(vinyl)tin produced the alkene 2 in 58% yield. The following hydroboration-oxidation reaction of 2 gave the hydroxyethyl compound 3 in 86% yield, which was then converted to 4 by reaction with DAST. 5 was prepared in good yield by a similar method.

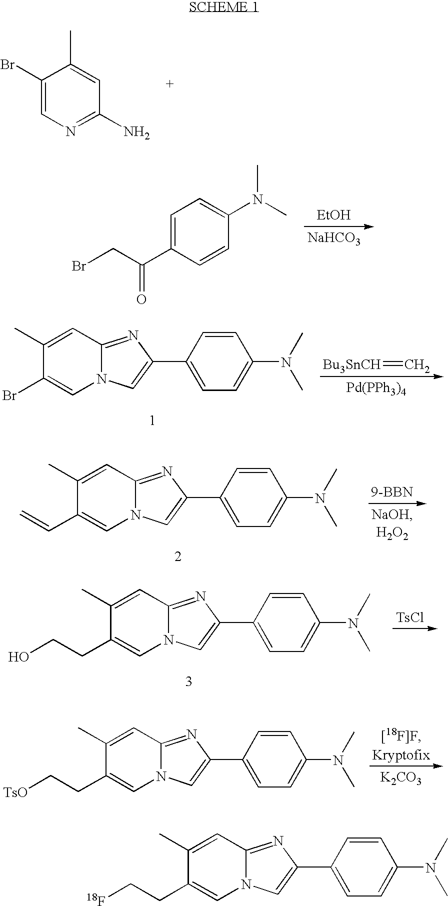

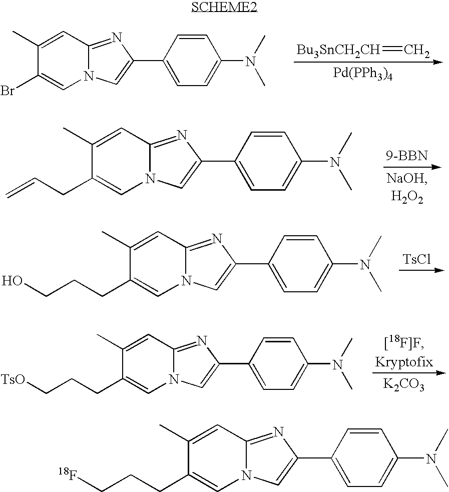

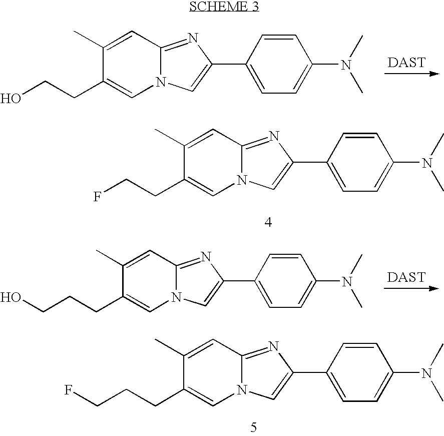

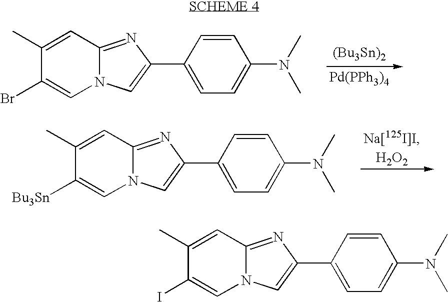

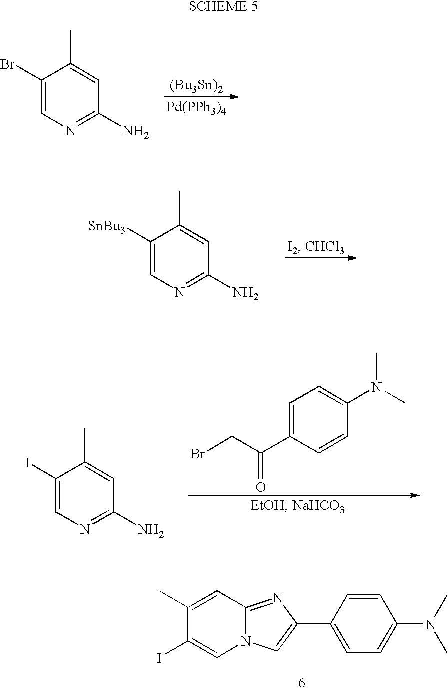

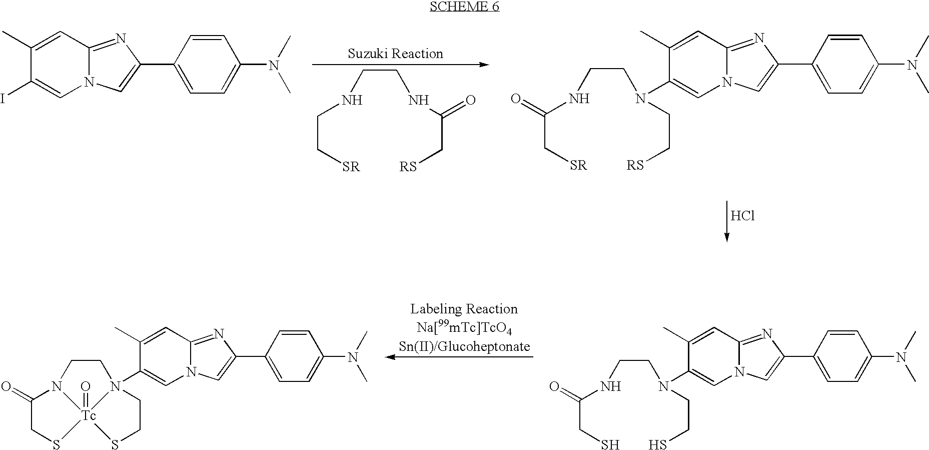

Schemes 7 through 11 depict synthetic routes for 7- or 8-substituted phenyl-imidazo[1,2-a][1,2-b]diazepine derivatives of the present invention.

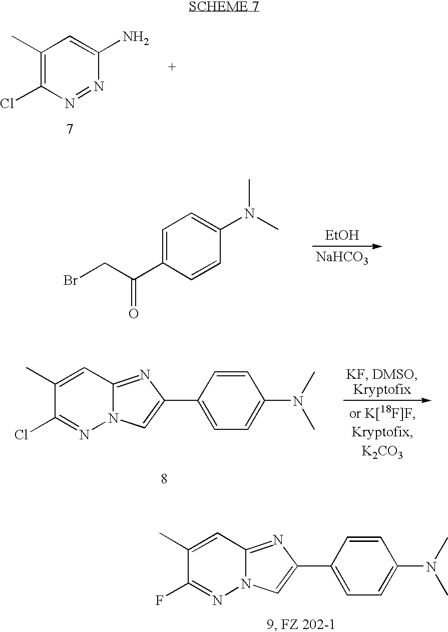

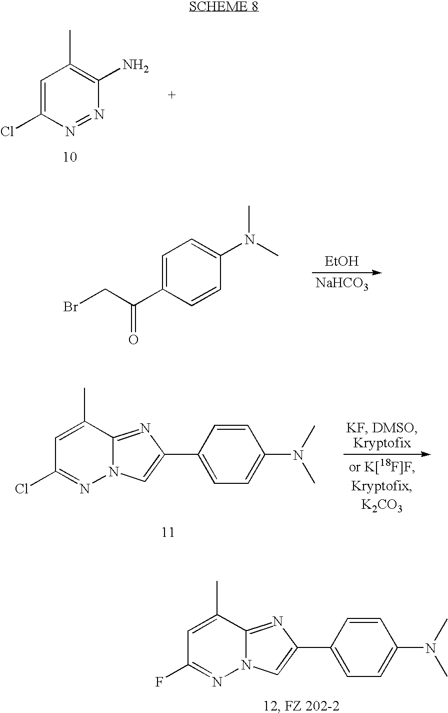

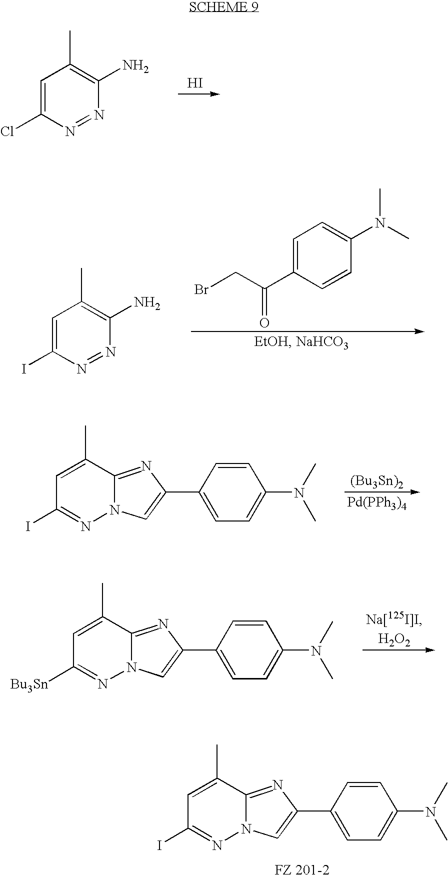

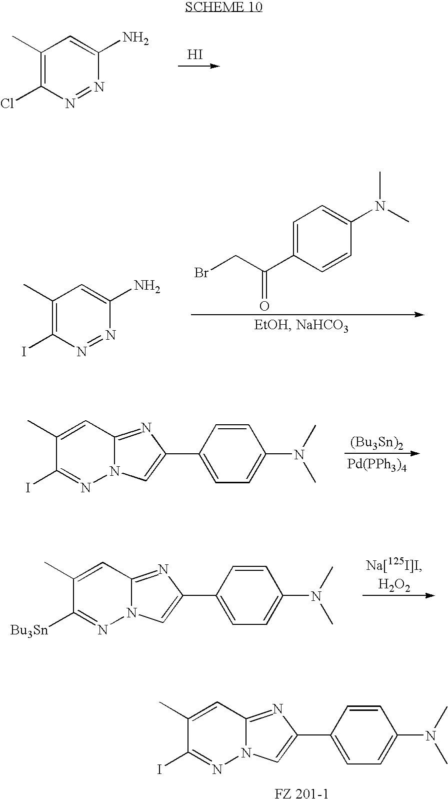

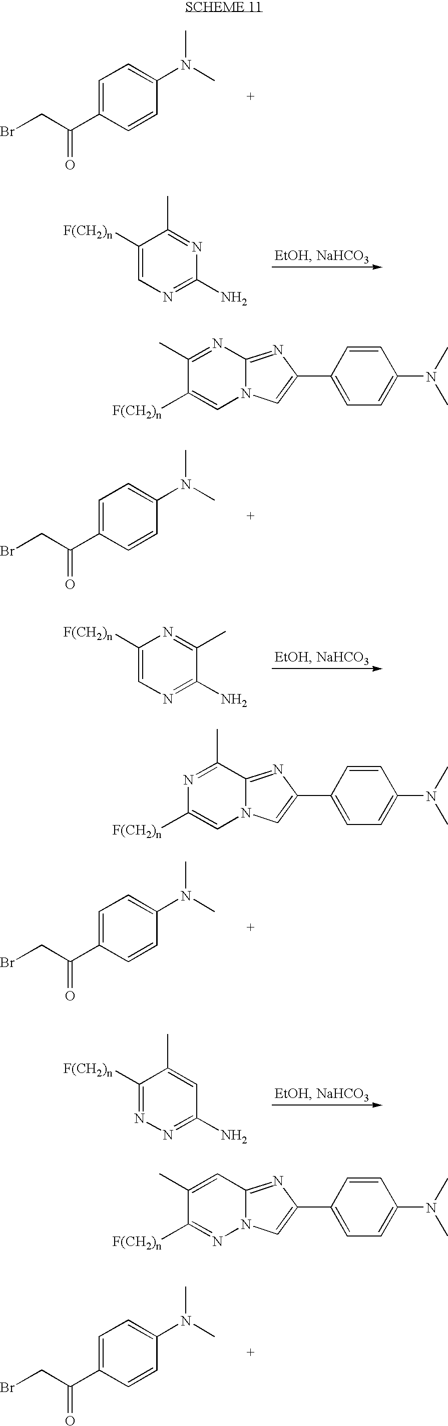

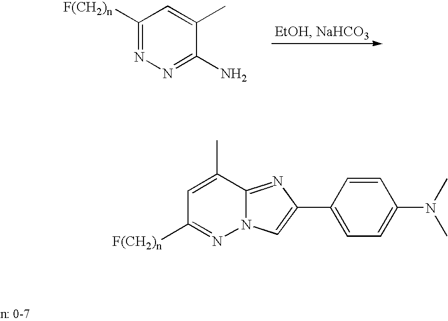

Schemes 12 through 14 are directed to thiophenyl or furanyl-imidazo[1,2-a]pyridine derivatives of the present invention.

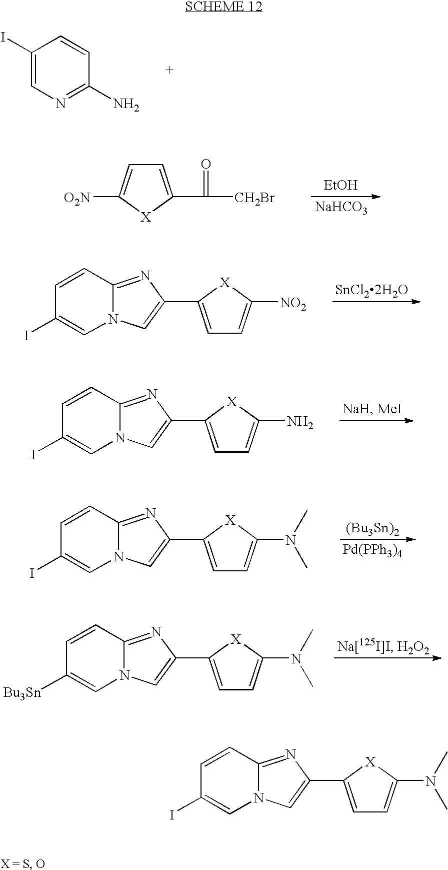

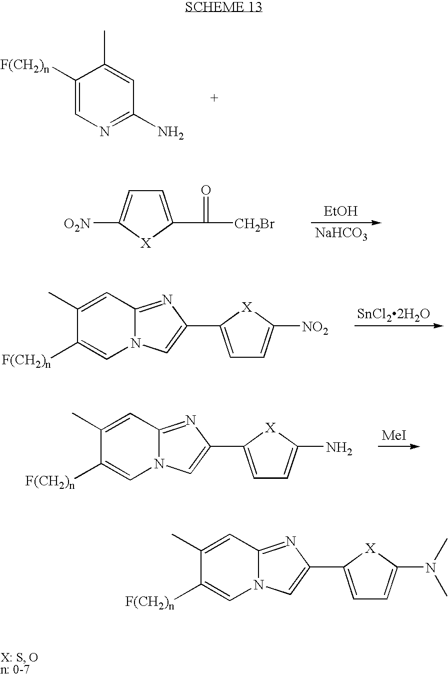

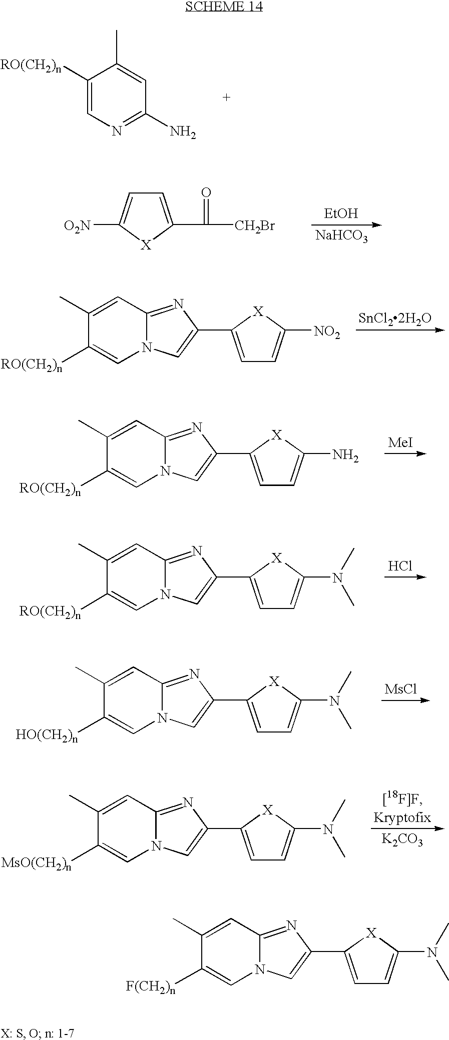

Schemes 15 through 19 depict synthetic routes for synthesis and parallel synthesis of thiophenyl, pyridyl, or furanyl-imidazo[1,2-b]pyridizine derivatives of the present invention.

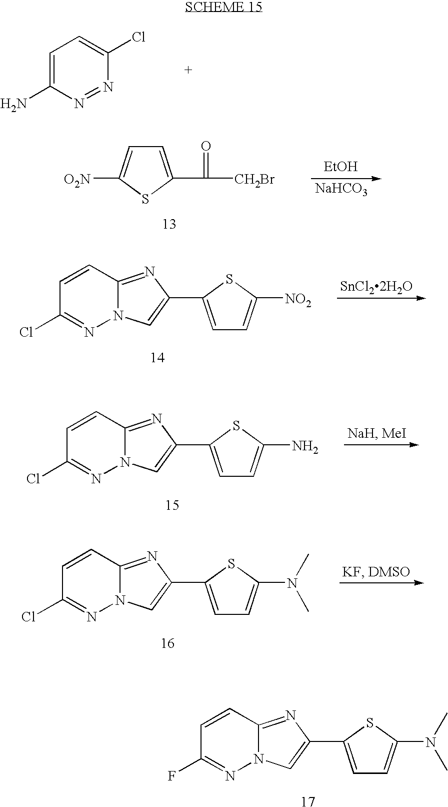

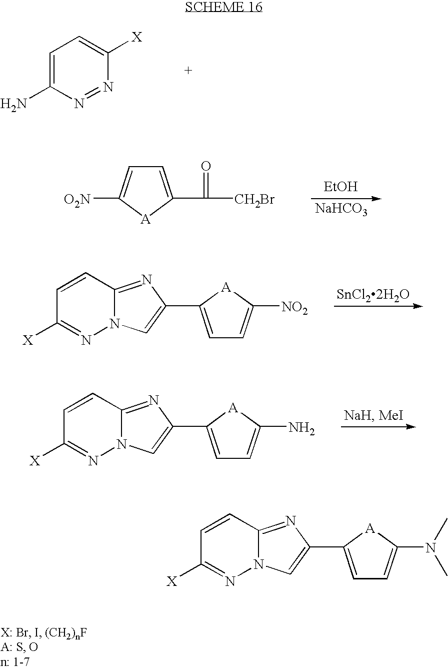

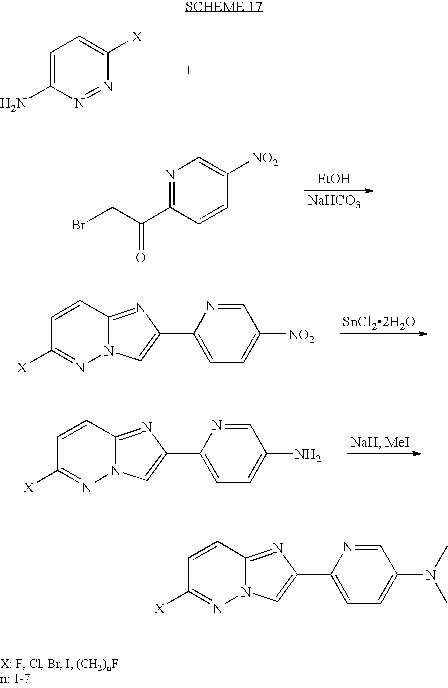

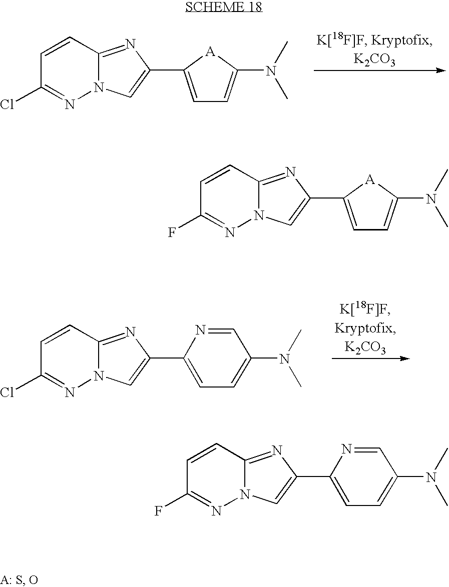

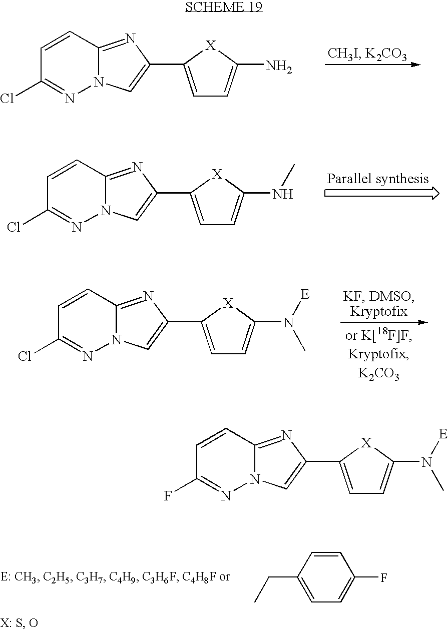

The present invention also includes stereoisomers as well as optical isomers, e.g. mixtures of enantiomers as well as individual enantiomers and diastereomers which arise as a consequence of structural asymmetry.

The compounds of Formula I, II or III may also be solvated, especially hydrated. Hydration may occur during manufacturing of the compounds or compositions comprising the compounds, or the hydration may occur over time due to the hygroscopic nature of the compounds. In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

When the compounds of the invention are to be used as imaging agents, they must be labeled with suitable radioactive halogen isotopes such as ¹²³I, ¹³¹I, ¹⁸F, ⁷⁶Br, and ⁷⁷Br. The radiohalogenated compounds of this invention can easily be provided in kits with materials necessary for imaging amyloid deposits. For example, a kit can contain a final product labeled with an appropriate isotope ready to use for imaging or a penultimate product (e.g. compounds of formula I having Sn(alkyl)₃ at the X position) and a label (e.g. K[¹⁸F]F) with reagents such that a final product can be made at the site or time of use.

In the first step of the present method of imaging, a labeled compound of Formula I, II or III is introduced into a tissue or a patient in a detectable quantity. The compound is typically part of a pharmaceutical composition and is administered to the tissue or the patient by methods well known to those skilled in the art. For example, the compound can be administered either orally, rectally, parenterally (intravenous, by intramuscularly or subcutaneously), intracistemally, intravaginally, intraperitoneally, intravesically, locally (powders, ointments or drops), or as a buccal or nasal spray.

In an imaging method of the invention, the labeled compound is introduced into a patient in a detectable quantity and after sufficient time has passed for the compound to become associated with amyloid deposits, the labeled compound is detected noninvasively inside the patient. In another embodiment of the invention, a labeled compound of Formula I, II or III is introduced into a patient, sufficient time is allowed for the compound to become associated with amyloid deposits, and then a sample of tissue from the patient is removed and the labeled compound in the tissue is detected apart from the patient. Alternatively, a tissue sample is removed from a patient and a labeled compound of Formula I, II or III is introduced into the tissue sample. After a sufficient amount of time for the compound to become bound to amyloid deposits, the compound is detected. The term “tissue” means a part of a patient's body. Examples of tissues include the brain, heart, liver, blood vessels, and arteries. A detectable quantity is a quantity of labeled compound necessary to be detected by the detection method chosen. The amount of a labeled compound to be introduced into a patient in order to provide for detection can readily be determined by those skilled in the art. For example, increasing amounts of the labeled compound can be given to a patient until the compound is detected by the detection method of choice. A label is introduced into the compounds to provide for detection of the compounds.

The administration of the labeled compound to a patient can be by a general or local administration route. For example, the labeled compound may be administered to the patient such that it is delivered throughout the body. Alternatively, the labeled compound can be administered to a specific organ or tissue of interest. For example, it is desirable to locate and quantitate amyloid deposits in the brain in order to diagnose or monitor the progress of Alzheimer's disease in a patient.

Those skilled in the art are familiar with determining the amount of time sufficient for a compound to become associated with amyloid deposits. The amount of time necessary can easily be determined by introducing a detectable amount of a labeled compound of Formulae I-III into a patient and then detecting the labeled compound at various times after administration.

Those skilled in the art are familiar with the various ways to detect labeled compounds. For example, magnetic resonance imaging (MRI), positron emission tomography (PET), or single photon emission computed tomography (SPECT) can be used to detect radiolabeled compounds. The label that is introduced into the compound will depend on the detection method desired. For example, if PET is selected as a detection method, the compound must possess a positron-emitting atom, such as ¹¹C or ¹⁸F.

The radioactive diagnostic agent should have sufficient radioactivity and radioactivity concentration which can assure reliable diagnosis. For instance, in case of the radioactive metal being technetium-99m (“Tc-99m complexed compounds”), it may be included usually in an amount of 0.1 to 50 mCi in about 0.5 to 5.0 ml at the time of administration. The amount of a compound of Formulae I-III may be such as sufficient to form a stable chelate compound with the radioactive metal.

The inventive compound as a radioactive diagnostic agent is sufficiently stable, and therefore it may be immediately administered as such or stored until its use. When desired, the radioactive diagnostic agent may contain any additive such as pH controlling agents (e.g., acids, bases, buffers), stabilizers (e.g., ascorbic acid) or isotonizing agents (e.g., sodium chloride). The imaging of amyloid deposits can also be carried out quantitatively so that the amount of amyloid deposits can be determined.

Preferred compounds for imaging include a radioisotope such as ¹²³I, ¹²⁴I, ¹²⁵I, ¹³¹I, ¹⁸F, ⁷⁶Br, ⁷⁷Br or ¹¹C.

The inventive compounds are particularly useful for imaging amyloid deposits in vivo. One of the key prerequisites for an in vivo imaging agent of the brain is the ability to cross the intact blood-brain barrier after a bolus intravenous injection. The compounds disclosed herein possess a core ring system comprised of various substituted, fused 5- and 6-member aromatic rings. Several compounds of this invention contain a benzothiazole core and are derivatives of thioflavins. These compounds contain no quaternary ammonium ion, therefore, they are relatively small in size, neutral and lipophilic.

Another aspect of the invention is a method of inhibiting amyloid plaque aggregation. The present invention also provides a method of inhibiting the aggregation of amyloid proteins to form amyloid deposits, by administering to a patient an amyloid inhibiting amount of a compound of the above Formula I, II or III. Those skilled in the art understand how to determine an amyloid inhibiting amount by simply administering a compound of Formula I, II or III to a patient in increasing amounts until the growth of amyloid deposits is decreased or stopped. The rate of growth can be assessed using imaging as described above or by taking a tissue sample from a patient and observing the amyloid deposits therein. The compounds of the present invention can be administered to a patient at dosage levels in the range of about 0.1 to about 1,000 mg per day. For a normal human adult having a body weight of about 70 kg, a dosage in the range of about 0.01 to about 100 mg per kilogram of body weight per day is sufficient. The specific dosage used, however, can vary. For example, the dosage can depend on a number of factors including the requirements of the patient, the severity of the condition being treated, and the pharmacological activity of the compound being used. The determination of optimum dosages for a particular patient is well known to those skilled in the art.

The examples presented below are intended to illustrate particular embodiments of the present invention and are not intended to limit the scope of the specification, including the claims in any manner.

### EXAMPLES

### 2-(4′-Dimethylaminophenyl)-6-(2′-fluoroethyl)-7-methylimidazo[1,2-a]pyridine, (4)

### 6-Bromo-2-(4′-dimethylaminophenyl)-7-methylimidazo[1,2-a]pyridine (1)

A mixture of 2-bromo-4′-dimethylaminoacetophenone [Diwu, Z. et el. Tetrahedron Lett., 39:4987-4990 (1998)] (1.23 g, 5.08 mmol) and 2-amino-4-methyl-5-bromopyridine (0.95 g, 5.08 mmol) in EtOH (50 mL) was stirred under reflux for 2 h. NaHCO₃ (635 mg) was added after the mixture was cooled. The resulting mixture was stirred under reflux for 6 h. The mixture was cooled and filtered to give 820 mg of product (49% yield). ¹H NMR (CD₂Cl₂, 400 MHz): δ 8.32 (s, 1H), 7.78 (d, J=9.0 Hz, 2H), 7.70 (s, 1H), 7.40 (s, 1H), 6.78 (d, J=8.8 Hz, 2H), 3.00 (s, 6H). 2.45 (s, 3H). HRMS: m/z calcd for C₁₇H₁₉N₃O (M⁺+H) 281.1528, found 281.1532. Anal. 1, (C₁₆H₁₆BrN₃).

### 2-(4′-Dimethylaminophenyl)-6-ethenyl-7-methylimidazo[1,2-a]pyridine (2)

To a solution of compound 1 (190 mg, 0.576 mmol) in toluene (12 mL) were added tributyl(vinyl)tin (0.84 mL, 2.88 mmol) and Pd(PPh₃)₄ (49 mg). The reaction mixture was stirred at 100° C. for 20 h. The toluene was removed under reduced pressure, and the black residue was purified by flash chromatography (hexane:ethyl acetate=1:1) to afford a yellow solid product (92 mg, 58%). ¹H NMR (CD₂Cl₂, 400 MHz): δ 8.16 (s, 1H), 7.78 (d, J=9.1 Hz, 2H), 7.71 (s, 1H), 7.27 (s, 1H), 6.82 (dd, J=10.4, 16.8 Hz, 1H), 6.77 (d, J=9.0 Hz, 2H), 5.59 (dd, J=1.3, 7.2 Hz, 1H), 5.30 (dd, J=1.3, 13.6 Hz, 1H), 2.99 (s, 6H), 2.38 (s, 3H). Anal. 2, (C₁₈H₁₉N₃).

### 2-(4′-Dimethylaminophenyl)-6-(2′-hydroxyethyl)-7-methylimidazo[1,2-a]pyridine (3)

To a solution of compound 2 (90 mg, 0.325 mmol) in THF (5 mL) was added 9-BBN (0.5 M in THF, 2.6 mL, 1.3 mmol). After being stirred at room temperature for 24 h, the mixture was cooled to 0° C. and added 3 N NaOH (1.3 mL), followed by 30% H₂O₂ (1.3 mL). The reaction mixture was warmed to room temperature and stirred overnight. The solvent was removed in vacuo and the aqueous solution was extracted with CH₂Cl₂. The combined organic layers were dried over MgSO₄ and solvent was removed under reduced pressure. Purification by flash chromatography (ethyl acetate: methanol=10:1) afforded a pale yellow solid product (82 mg, 86%). ¹H NMR (CD₂Cl₂, 400 MHz): δ 7.97 (s, 1H), 7.79 (d, J=8.8 Hz, 2H), 7.63 (s, 1H), 7.32 (s, 1H), 6.78 (d, J=8.8 Hz, 2H), 3.86 (t, J=6.2 Hz, 2H), 3.00 (s, 6H). 2.84 (t, J=6.2 Hz, 2H), 2.37 (s, 3H). Anal. 3, (C₁₈H₂₁N₃O).

### 2-(4′-Dimethylaminophenyl)-6-(2′-fluoroethyl)-7-methylimidazo[1,2-a]pyridine (4)

To a solution of (diethylamino)sulfur trifluoride (0.158 mL, 1.2 mmol) in anhydrous CH₂Cl₂ (2 mL) at −78° C. was added 3 (82 mg, 0.28 mmol) in CH₂Cl₂ (4 mL). The reaction mixture was stirred at this temperature for 1.5 h, then warmed to room temperature and the stirring was continued for 1 h. The reaction mixture was diluted with CH₂Cl₂ and washed with a saturated aqueous NaHCO₃. The organic layer was dried over MgSO₄ and evaporated. Purification by flash chromatography (hexane:ethyl acetate=1:4) gave a pale yellow solid product (26 mg, 31%). ¹H NMR (CD₂Cl₂, 400 MHz): δ 7.97 (s, 1H), 7.78 (d, J=9.0 Hz, 2H), 7.76 (s, 1H), 7.39 (s, 1H), 6.77 (d, J=9.0 Hz, 2H), 4.72 (t, J=6.2 Hz, 1H), 4.60 (t, J=6.2 Hz, 1H), 3.04 (t, J=6.4 Hz, 1H). 2.99 (s, 6H), 2.98 (t, J=6.4 Hz, 1H), 2.37 (s, 3H). Anal. 4, (C₁₈H₂₀N₃F).

### Example 2

### 2-(4′-Dimethylaminophenyl)-6-fluoro-7-methylimidazo[1,2-b]pyridazine, (9)

### 2-(4′-Dimethylaminophenyl)-6-fluoro-8-methylimidazo[1,2-b]pyridazine, (12)

### 3-Amino-6-chloro-4-methylpyridazine (7) and 3-Amino-6-chloro-4-methylpyridazine (10)

[Linholter, S., et al, Acta Chem. Scand. 15: 1660-1666 (1961)]. To a solution of 3,6-dichloro-4-methyl-pyridazine (200 mg) in ethanol (3 ml) was added liquid ammonia (3 ml). The reaction mixture was heated at 120° C. in a sealed tube for 12 h. After cooling to room temperature, methanol and ammonia were evaporated. The residue was purified by flash chromatography (ethyl acetate) to afford 153 mg of mixture of 7 and 10 (1:1), (87%), which was used without any further separation.

### 2-(4′-Dimethylaminophenyl)-6-chloro-7-methylimidazo[1,2-b]pyridazine, (8) and

### 2-(4′-Dimethylaminophenyl)-6-chloro-8-methylimidazo[1,2-b]pyridazine, (11)

A mixture of product from previous reaction (143 mg, 1 mmol) and 2-bromo-4′-dimethylaminoacetophenone (242 mg, 1 mmol) in EtOH (12 mL) was stirred under reflux for 2 h. NaHCO₃ (125 mg) was added after the mixture was cooled. The resulting mixture was stirred under reflux for 6 h. Solvent was removed, and the residue was purified by flash chromatography (hexane:ethyl acetate=1:1) to provide 8 (less polar compound) (54 mg, 38%) and 11 (66 mg, 46%) as yellow solids. 8: ¹H NMR (CD₂Cl₂, 400 MHz): δ 8.06 (s, 1H), 7.82 (d, J=9.0 Hz, 2H), 6.85 (d, J=1.1 Hz, 1H), 6.78 (d, J=8.8 Hz, 2H), 3.00 (s, 6H), 2.65 (d, J=1.1 Hz, 3H). HRMS: m/z calcd for C₁₅H₁₅N₄Cl (M⁺+H) 287.1058, found 287.1056.11: ¹H NMR (CD₂Cl₂, 400 MHz): δ 8.04 (s, 1H), 7.80 (d, J=9.0 Hz, 2H), 7.69 (d, J=1.1 Hz, 1H), 6.78 (d, J=9.0 Hz, 2H), 3.00 (s, 6H), 2.42 (d, J=1.1 Hz, 3H). HRMS: m/z calcd for C₁₅H₁₅N₄Cl (M⁺+H) 287.1058, found 287.1058.

### 2-(4′-Dimethylaminophenyl)-6-fluoro-7-methylimidazo[1,2-b]pyridazine, (9)

A solution of 8 (20 mg, 0.07 mmol), KF (12 mg, 0.21 mmol), and Kryptofix (37 mg, 0.1 mmol) in DMSO (2 mL) was heated at 130° C. in a screw-cap tube for 2 h. Additional portions of KF and Kryptofix were added, and heating continued for a total of 16 h. The entire reaction mixture was submitted to flash chromatography (hexane:ethyl acetate=1:1) to provide gave a yellow solid product (4 mg, 21%). ¹H NMR (CD₂Cl₂, 400 MHz): δ 8.00 (s, 1H), 7.81 (d, J=9.2 Hz, 2H), 6.79 (d, J=9.2 Hz, 2H), 6.66 (d, J=1.1 Hz, 1H), 3.00 (s, 6H), 2.69 (d, J=0.9 Hz, 3H). HRMS: m/z calcd for C₁₅H₁₅N₄F (M⁺+H) 271.1353, found 271.1352.

### 2-(4′-Dimethylaminophenyl)-6-fluoro-8-methylimidazo[1,2-b]pyridazine, (12)

The procedure described above to prepare 9 was employed to give 43% of product 12 from 11 (20 mg, 0.07 mmol), KF (12 mg, 0.21 mmol), and Kryptofix (37 mg, 0.1 mmol). ¹H NMR (CD₂Cl₂, 400 MHz): δ 7.96 (s, 1H), 7.77 (d, J=9.0 Hz, 2H), 7.71 (dt, J=1.1, 8.8 Hz, 1H), 6.78 (d, J=9.1 Hz, 2H), 3.00 (s, 6H), 2.35 (t, J=1.1 Hz, 3H). HRMS: m/z calcd for C₁₅H₁₅N₄F (M⁺+H) 271.1353, found 271.1353.

### Example 3

### 2-(4′-Dimethylaminothiophenyl)-6-fluoroimidazo[1,2-b]pyridazine, (17)

### 2-Bromoacetyl-5-nitrothiophene, (13)

Copper (II) bromide (1.11 g, 5 mmol) in ethyl acetate (20 ml) was heated to reflux, then a solution of 2-acetyl-5-nitrothiophene (510 mg, 3 mmol) in chloroform (10 ml) was added. After the mixture had been refluxing for 20 h, the liquid had become amber colored and the formation of HBr had ceased. The mixture was cooled and the precipitated copper (I) bromide was filtered off. The filtrate was stirred for 10 min with charcoal, filtered and evaporated to give product 540 mg (72%). ¹H NMR (CD₂Cl₂, 400 MHz): δ 7.92 (d, J=4.4 Hz), 7.69 (d, J=4.2 Hz), 4.41 (s, 2H).

### 6-Chloro-2-(4′-nitrothiophenyl)-imidazo[1,2-b]pyridazine (14)

A mixture 2-bromoacetyl-5-nitrothiophene (190 mg, 0.76 mmol) and 3-amino-6-chloropyridazine (98 mg, 0.76 mmol) in EtOH (4 mL) was stirred under reflux for 2 h. NaHCO₃ (115 mg) was added after the mixture was cooled. The resulting mixture was stirred under reflux for 6 h. The mixture was cooled and filtered to give 120 mg of product as brown solid product (56% yield). ¹H NMR (CD₂Cl₂, 400 MHz): δ 8.28 (s, 1H), 7.93 (d, J=4.4 Hz, 1H), 7.92 (d, J=9.5 Hz, 1H), 7.40 (d, J=4.4 Hz, 1H), 7.15 (d, J=9.5 Hz, 1H). HRMS: m/z calcd for C₁₀H₆N₄O₂CIS (M⁺+H) 280.9893, found 280.9891.

### 2-(4′-Aminothiophenyl)-6-chloroimidazo[1,2-b]pyridazine(15)

Conc. HCl soln. (1.37 ml) was added dropwise to a mixture of 14 (116 mg, 0.41 mmol) and SnCl₂.2H₂O (837 mg, 3.3 mmol) in 95% EtOH (5 ml) at room temperature. Sufficient cooling was necessary to keep the reaction temperature under 35° C. The mixture was stirred at 35° C. for 1 h. The EtOH was evaporated and the aqueous layer washed with hexane. The aqueous layer was neutralized with 1N NaOH to pH 9 and the mixture was extracted several times with AcOEt. The combined organic layers were dried over MgSO₄ and solvent was removed under reduced pressure. Purification by flash chromatography (hexane:ethyl acetate=1:1) afforded a brown solid product (59 mg, 57%). ¹H NMR (DMSO-d₆, 400 MHz): 8.46 (s, 1H), 8.01 (d, J=9.3 Hz, 1H), 7.23 (d, J=9.3 Hz, 1H), 7.12 (d, J=3.8 Hz, 1H), 5.91 (s, 2H), 5.86 (d, J=3.8 Hz, 1H). HRMS: m/z calcd for C₁₀H₈N₄CIS (M⁺+H) 251.0153, found 251.0149.

### 6-Chloro-2-(4′-dimethylaminothiophenyl)-imidazo[1,2-b]pyridazine (16)

To a suspension of NaH (60% oil dispersion, 32 mg, 0.8 mmol) in DMSO (3 ml) was added 15 (50 mg, 0.2 mmol) at room temperature. After 1 h MeI (62 μL, 1 mmol) was added to the mixture. The mixture was stirred for another 1 h at room temperature and then the entire reaction mixture was submitted to flash chromatography (hexane:ethyl acetate=1:1) to provide gave a yellow solid product (12 mg, 21%). ¹H NMR (DMSO-d6, 400 MHz): 8.51 (s, 1H), 8.01 (d, J=9.5 Hz, 1H), 7.28 (d, J=3.8 Hz, 1H), 7.24 (d, J=9.4 Hz, 1H), 5.91 (d, J=3.8 Hz, 1H), 2.89 (s, 6H). HRMS: m/z calcd for C12H12N4CIS (M++H) 279.0466, found 279.0461.

### 2-(4′-Dimethylaminothiophenyl)-6-fluoroimidazo[1,2-b]pyridazine (17)

A mixture of 16 (12 mg, 0.043 mmol), KF (15 mg, 0.26 mmol), and Kryptofix 222 (16 mg, 0.043 mmol) in DMSO (1 ml) was stirred in a sealed vial at 160-170° C. for 20 h. The entire reaction mixture was submitted to PTLC(hexane:ethyl acetate=1:1) to provide a yellow solid product (3 mg, 26%). ¹H NMR (DMSO-d₆, 400 MHz): 8.44 (s, 1H), 8.13 (t, J=8.7 Hz, 1H), 7.25 (d, J=3.8 Hz, 1H), 7.14 (d, J=9.7 Hz, 1H), 5.90 (d, J=3.8 Hz, 1H), 2.89 (s, 6H). HRMS: m/z calcd for C₁₂H₁₂N₄FS (M⁺+H) 263.0761, found 263.0757.

### Example 4

### Preparation of Radiofluorinated Ligand: [¹⁸F]-9

The compound, [¹⁸F]-9, was prepared by nucleophilic substitution of the corresponding chloro-precursor 8. No-carrier-added (NCA) [¹⁸F]-fluoride was produced at Emory University Hospital with a 11 MeV Siemens RDS 112 negative-ion cyclotron (Knoxyille, Tenn., USA) by the ¹⁸O (p, n) ¹⁸F reaction using [¹⁸O] enriched water (>95 atom %). The radiosynthesis of [¹⁸F]-9 was performed in a chemical process control unit (CPCU) obtained from CTI, Inc. (Knoxyille, Tenn., USA). NCA aqueous [¹⁸F]-fluoride (0.8 mL) delivered to the trap/release cartridge (DW-TRC, D&W, Inc.) was released with 0.6 mL of water containing 0.9 mL of potassium carbonate as K[¹⁸F]F and added to a Pyrex vessel which contained 5 mg of Kryptofix 2.2.2 in 1 mL of CH₃CN. The water was evaporated using a stream of nitrogen at 110° C. and co-evaporated to dryness with CH₃CN (3 mL). The chloro-precursor 8 (5 mg in 0.6 mL of DMSO) was then added to the dried K[¹⁸F]F and the solution was heated at 150° C. for 15 min and then cooled to room temperature. Ether (3×3 mL) was added and the contents of the vessel were transferred through the silica SepPak to a V-vial. After evaporation of ether, the residue was diluted with HPLC mobile phase (500 μL) for direct injection onto an HPLC column (waters, Xterra Prep RP₁₈ 5 μm, 19×100 mm) eluted with MeOH:H₂O:Et₃N=62:38:0.1 at 8.0 mL/min, with eluate monitored for radioactivity. The radioactive fractions with the same retention time as the respective reference ligand (t_{R}=19.0 min) were collected, combined and diluted with double volume of water. The solution was passed through a Waters C₁₈ SepPak cartridge which was washed with saline (0.9% NaCl, 40 mL) and ethanol (0.5 mL). The radioactive product was washed out of the cartridge by absolute ethanol (1.5 mL) into a sterile empty vial containing 3.5 mL of saline. The resulting solution was transferred under argon pressure through a Millipore filter (pore size 1.0 μm) followed by a smaller one (pore size 0.2 μm), to a 30 mL sterile vial containing 10 mL of saline and is ready for PET study.

The final product was analyzed on a analytical HPLC (Waters Nova-Pak C₁₈ 3.9×150 mm) eluted with MeOH:H₂O:Et₃N=70:30:0.1 at 1.0 mL/min (t_{R}=7.5 min). Radioactivity and absorbance (254 nm) were monitored to confirm the radiochemical purity of [¹⁸F]-9 and to measure its specific radioactivity.

### Example 5

### Partition Coefficient Determination

Measurement of distribution coefficient of [¹⁸F]tracer was performed based on the method described by Wilson et al. [Labelled Compd. Radiopharm., 42:1277-1288 (1999)] using 0.02 M sodium phosphate buffer at pH 7.4 and 1-octanol. Basically, the test tubes containing ˜20 μCi of the radiotracer and 2 mL each of 1-octanol and phosphate buffer were vortexed for 10 min at room temperature and then centrifuged for 5 min. Samples (0.5 mL) from 1-octanol and buffer layers were counted in a Packard Cobra II auto-gamma counter (Perkin-Elmer, Downers Grove, Ill.) and decay corrected. The measurement was repeated three times. The log P_{7.4} value was calculated as follows: log P_{7.4} log [counts in octanol phase/counts in buffer phase].

### Example 6

### Binding Assays Using Human AD Brain Tissues by Quantitative Autoradiography

Postmortem human cerebral cortical tissue from the frontal lobe was obtained from the Center for Neurodegenerative Disease at Emory University (Atlanta, Ga.). Fresh-frozen tissue sections were cut at a thickness of 20-25 μm and thaw-mounted onto gelatin-coated glass slides. The sections were then air-dried and stored at −80° C. until used. Prepared sections were thawed and incubated at room temperature in 0.05M Tris-HCl buffer, pH 7.7 with 10% ethanol containing 0.02 nM [¹²⁵I] IMPY. The radioligand was displaced with increasing concentrations (0.1 nM-5 μM) of cold inhibitor (in 200 μl of 100% ethanol). Nonspecific binding was determined in the presence of 5 μM thioflavin-T (THFT). Borosilicate glass tubes were used for the incubation containers to minimize hydrophobic adsorption to the walls. After 5 hours, the sections were washed with 100% ethanol for 30 minutes at room temperature and allowed to air-dry. The radiolabeled sections and ¹⁴C-plastic standards (calibrated for ¹²⁵I, American Radiolabeled Chemicals, Inc., St. Louis, Md.) were apposed to autoradiographic film (Biomax MS, Eastman Kodak, Rochester, N.Y.) for 24 hours. The resulting autoradiograms were digitized using an Epson 1680 Scanner with transparency unit and analyzed densitometrically with AIS software (Imaging Research, St. Catherines, Ontario) to determine binding density. Binding curves and corresponding Kᵢ valves were generated using non-linear regression with GraphPad Prism software.

### Example 7

### In Vivo Brain Uptake Study of New Probes in Rhesus Monkey

PET study was performed in a male rhesus monkey weighing 6-10 kg using a Concorde MicroPET P4 imaging system. The animal was initially anesthetized with an intramuscular injection of Telazol (3 mg/kg), intubated, and then maintained on a 1% isoflurane/5% oxygen gas mixture throughout the imaging session. The monkey was placed in the PET scanner and the head was immobilized. Blood pressure, heart and respiratory rates, and expired CO₂ and oxygen saturation levels were monitored continuously during the PET study. A transmission scan was obtained with a germanium-68 source prior to the PET study for attenuation correction of the emission data. Brain emission scans were performed following the intravenous administration of [¹⁸F]tracer (5 mCi). Serial dynamic transaxial images were acquired for a total of 120 min and then binned for analysis. Emission data acquired were subject to iterative reconstruction (OSEM, two iterations, 40 subsets) with no pre- or postfiltering to provide images with an isotropic resolution of 3 mm fwhm. For generation of time-activity curves, regions of interest (ROIs) were drawn manually based on the anatomical landmarks visible in reconstructed PET images using ASIPro software (Concorde, Knoxyille, Tenn.).

The foregoing exemplary descriptions and the illustrative preferred embodiments of the present invention have been explained in the drawings and described in detail, with varying modifications and alternative embodiments being taught. While the invention has been so shown, described and illustrated, it should be understood by those skilled in the art that equivalent changes in form and detail may be made therein without departing from the true spirit and scope of the invention, and that the scope of the invention is to be limited only to the claims except as precluded by the prior art. Moreover, the invention as disclosed herein, may be suitably practiced in the absence of the specific elements which are disclosed herein.

When a group of substituents is disclosed herein, it is understood that all individual members of that group and all subgroups, including any isomers and enantiomers of the group members and classes of compounds that can be formed using the substituents are disclosed separately. When a Markush group or other grouping is used herein, all individual members of the group and all combinations and subcombinations possible of the group are intended to be individually included in the disclosure. When a compound is described herein such that a particular isomer or enantiomer of the compound is not specified, for example, in a formula or in a chemical name, that description is intended to include each isomers and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Specific names of compounds are intended to be exemplary, as it is known that one of ordinary skill in the art can name the same compounds differently.

Many of the molecules disclosed herein contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application.

Every formulation or combination of components described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure.

All patents and publications mentioned in the specification are indicative of the levels of skill of those skilled in the art to which the invention pertains. References cited herein are incorporated by reference herein in their entirety to indicate the state of the art as of their filing date and it is intended that this information can be employed herein, if needed, to exclude specific embodiments that are in the prior art. For example, when a compound is claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, reagents, solid substrates, synthetic methods, purification methods, and analytical methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The exact formulation, route of administration and dosage can be chosen by the individual physician in view of the patient's condition (see e.g. Fingl et. al., in The Pharmacological Basis of Therapeutics, 1975, Ch. 1 p. 1).

It should be noted that the attending physician would know how to and when to terminate, interrupt, or adjust administration due to toxicity, or to organ dysfunctions, or other adverse reactions. Conversely, the attending physician would also know to adjust treatment to higher levels if the clinical response were not adequate (precluding toxicity). The magnitude of an administered dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above also may be used in veterinary medicine.

Depending on the specific conditions being treated and the targeting method selected, such agents may be formulated and administered systemically or locally. Techniques for formulation and administration may be found in Alfonso and Gennaro (1995). Suitable routes may include, for example, oral, rectal, transdermal, vaginal, transmucosal, or intestinal administration; parenteral delivery, including intramuscular, subcutaneous, or intramedullary injections, as well as intrathecal, intravenous, or intraperitoneal injections.

For injection, the agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

Use of pharmaceutically acceptable carriers to formulate the compounds herein disclosed for the practice of the invention into dosages suitable for systemic administration is within the scope of the invention. With proper choice of carrier and suitable manufacturing practice, the compositions of the present invention, in particular those formulated as solutions, may be administered parenterally, such as by intravenous injection. Appropriate compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a patient to be treated.

Agents intended to be administered intracellularly may be administered using techniques well known to those of ordinary skill in the art. For example, such agents may be encapsulated into liposomes, then administered as described above. Liposomes are spherical lipid bilayers with aqueous interiors. All molecules present in an aqueous solution at the time of liposome formation are incorporated into the aqueous interior. The liposomal contents are both protected from the external microenvironment and, because liposomes fuse with cell membranes, are efficiently delivered into the cell cytoplasm. Additionally, due to their hydrophobicity, small organic molecules may be directly administered intracellularly.

Pharmaceutical compositions suitable for use in the present invention include compositions wherein the active ingredients are contained in an effective amount to achieve the intended purpose. Determination of the effective amounts is well within the capability of those skilled in the art, especially in light of the detailed disclosure provided herein.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions, including those formulated for delayed release or only to be released when the pharmaceutical reaches the small or large intestine.

The pharmaceutical compositions of the present invention may be manufactured in a manner that is itself known, e.g., by means of conventional mixing, dissolving, granulating, dragee-making, levitating, emulsifying, encapsulating, entrapping or lyophilizing processes.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fafty oils, liquid paraffin, or liquid polyethylene glycols. In addition, stabilizers may be added.

Although the description herein contains many specificifies, these should not be construed as limiting the scope of the invention, but as merely providing illustrations of some of the embodiments of the invention. Thus, additional embodiments are within the scope of the invention and within the following claims. All references cited herein are hereby incorporated by reference to the extent that there is no inconsistency with the disclosure of this specification. Some references provided herein are incorporated by reference to provide details concerning sources of starting materials, additional starting materials, additional reagents, additional methods of synthesis, additional methods of analysis and additional uses of the invention.

1. A compound of formula I: 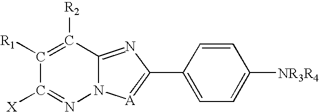
or a pharmaceutically acceptable salt thereof,
wherein R₁ and R₂ are each independently selected from the group consisting of: H, CH₃, CH₂(CH₂)ₙCH₃, CH₂(CH₂)ₙCH₂F, CH₂CH═CH(CH₂)ₙF, (CH₂)ₙCH═CH(CH₂)ₙI, OH, OCH₃, OCH₂(CH₂)ₙCH₃, OCH₂(CH₂)ₙCH₂F, OCH₂CH═CH(CH₂)ₙF, O(CH₂)ₙCH═CH(CH₂)ₙI, SH, SCH₃, SCH₂(CH₂)ₙCH₃, SCH₂(CH₂)ₙCH₂F, SCH₂CH═CH(CH₂)ₙF, S(CH₂)ₙCH═CH(CH₂)ₙI (n=0-7), aromatic, haloaromatic, heteroaromatic, haloheteroaromatic, cyclic, halogen, and halocyclic; X is selected from the group consisting of: F, Cl, Br, I, CH₂(CH₂)ₙCH₂F, CH₂CH═CH(CH₂)ₙF, (CH₂)ₙCH═CH(CH₂)ₙI, OH, OCH₃, OCH₂(CH₂)ₙCH₃, OCH₂(CH₂)ₙCH₂F, OCH₂CH═CH(CH₂)ₙF, O(CH₂)ₙCH═CH(CH₂)ₙI, SH, SCH₃, SCH₂(CH₂)ₙCH₃, SCH₂(CH₂)ₙCH₂F, SCH₂CH═CH(CH₂)ₙF, S(CH₂)ₙCH═CH(CH₂)ₙI (n=0-7), and Tc-99m complexed compounds; A is CH or N; R₃ and R₄ are independently selected from the group consisting of: hydrogen, C₁₋₄ alkyl, C₂₋₄ aminoalkyl, C₁₋₄ haloalkyl, and haloarylalkyl, or R₃ and R₄ are taken together with the nitrogen to which they are attached to form a 5- to 7-member heterocyclic ring optionally having O, S or NR₅ in said ring, where R₅ is hydrogen or C₁₋₄ alkyl, provided that both R₁ and R₂ are not both H when A is CH, and X is Br, I, F, ¹²⁵I, ¹³¹I, ¹²³I, ¹⁸F, ⁷⁶Br, ⁷⁷Br, haloalkyl, Sn(alkyl)₃ or -L-Ch, where L is —(CH₂)ₙ— or —(CH₂)ₙ—C(O)—, where n is 0 to 5 and Ch is a tetradentate ligand capable of complexing with a metal.

2. The compound of claim 1, wherein A is CH.

3. The compound of claim 1 having one of the formulas:

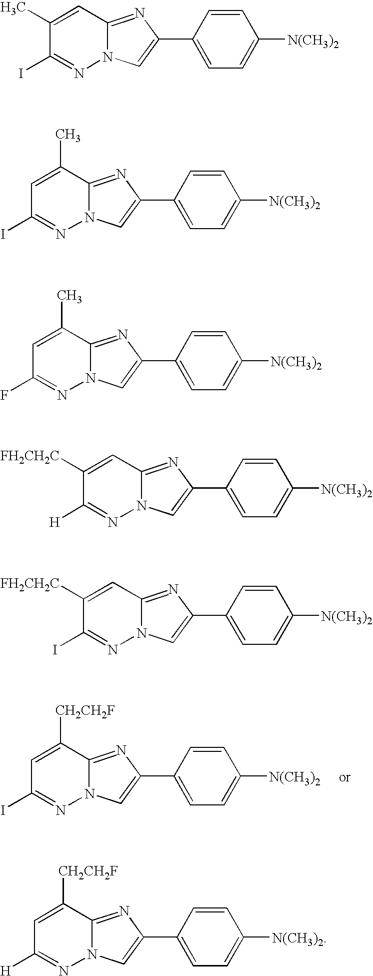

4. (canceled)

5. (canceled)

6. (canceled)

7. (canceled)

8. (canceled)

9. (canceled)

10. A compound of formula II: 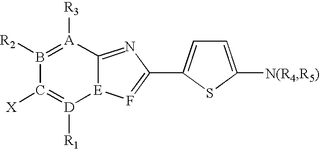
or a pharmaceutically acceptable salt thereof,
wherein R₁, R₂, R₃ are each independently selected from the group consisting of: H, CH₃, CH₂(CH₂)ₙCH₃, CH₂(CH₂)ₙCH₂F, CH₂CH═CH(CH₂)ₙF, (CH₂)ₙCH═CH(CH₂)ₙI, OH, OCH₃, OCH₂(CH₂)ₙCH₃, OCH₂(CH₂)ₙCH₂F, OCH₂CH═CH(CH₂)ₙF, O(CH₂)ₙCH═CH(CH₂)ₙI, SH, SCH₃, SCH₂(CH₂)ₙCH₃, SCH₂(CH₂)ₙCH₂F, SCH₂CH═CH(CH₂)ₙF, S(CH₂)ₙCH═CH(CH₂)ₙI, aromatic, haloaromatic, heteroaromatic, haloheteroaromatic, cyclic, halogen and halocyclic; R₄, R₅ are each independently selected from the group consisting of: H, CH₃, CH₂(CH₂)ₙCH₃, CH₂(CH₂)ₙCH₂F, CH₂CH═CH(CH₂)ₙF, (CH₂)ₙCH═CH(CH₂)ₙI, OH, OCH₃, OCH₂(CH₂)ₙCH₃, OCH₂(CH₂)ₙCH₂F, OCH₂CH═CH(CH₂)ₙF, O(CH₂)ₙCH═CH(CH₂)ₙI, SH, SCH₃, SCH₂(CH₂)ₙCH₃, SCH₂(CH₂)ₙCH₂F, SCH₂CH═CH(CH₂)ₙF, S(CH₂)ₙCH═CH(CH₂)ₙI, aromatic, haloaromatic, heteroaromatic, haloheteroaromatic, cyclic, and halocyclic, or R₄ and R₅ are taken together with the nitrogen to which they are attached to form a 5- to 7-member heterocyclic ring optionally having O, S or NR₆ in said ring, where R₆ is hydrogen or C₁₋₄ alkyl; X is selected from the group consisting of: F, Cl, Br, I, CH₂(CH₂)ₙCH₂F, CH₂CH═CH(CH₂)ₙF, (CH₂)ₙCH═CH(CH₂)ₙI, OH, OCH₃, OCH₂(CH₂)ₙCH₃, OCH₂(CH₂)ₙCH₂F, OCH₂CH═CH(CH₂)ₙF, O(CH₂)ₙCH═CH(CH₂)ₙI, SH, SCH₃, SCH₂(CH₂)ₙCH₃, SCH₂(CH₂)ₙCH₂F, SCH₂CH═CH(CH₂)ₙF, S(CH₂)ₙCH═CH(CH₂)ₙI (n=0-7), and Tc-99m complexed compounds; A, B, C, and D are each independently selected from the group consisting of: C, CH, N, N⁺O⁻; E and F are each independently selected from the group consisting of: C, CH and N.

11. The compound of claim 10, wherein A, B, and C are C; F is CH; and D and E are N.

12. The compound of claim 10 having one of the formulas:

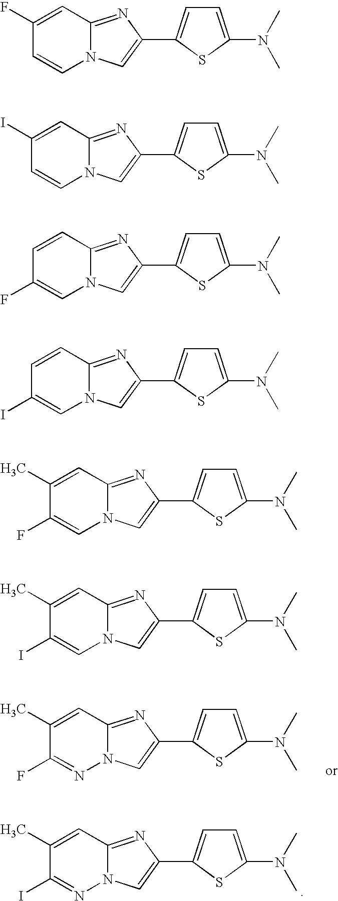

13. (canceled)

14. (canceled)

15. (canceled)

16. (canceled)

17. (canceled)

18. (canceled)

19. (canceled)

20. A compound of formula III: 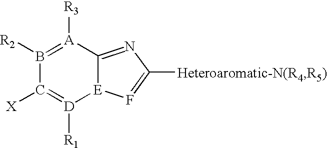
or a pharmaceutically acceptable salt thereof,
wherein R₁, R₂, R₃ are each independently selected from the group consisting of: H, CH₃, CH₂(CH₂)ₙCH₃, CH₂(CH₂)ₙCH₂F, CH₂CH═CH(CH₂)ₙF, (CH₂)ₙCH═CH(CH₂)ₙI, OH, OCH₃, OCH₂(CH₂)ₙCH₃, OCH₂(CH₂)ₙCH₂F, OCH₂CH═CH(CH₂)ₙF, O(CH₂)ₙCH═CH(CH₂)ₙI, SH, SCH₃, SCH₂(CH₂)ₙCH₃, SCH₂(CH₂)ₙCH₂F, SCH₂CH═CH(CH₂)ₙF, S(CH₂)ₙCH═CH(CH₂)ₙI (n=0-7), aromatic, haloaromatic, heteroaromatic, haloheteroaromatic, cyclic, halogen and halocyclic; R₄R₅ are each independently selected from the group consisting of: H, CH₃, CH₂(CH₂)ₙCH₃, CH₂(CH₂)ₙCH₂F, CH₂CH═CH(CH₂)ₙF, (CH₂)ₙCH═CH(CH₂)ₙI, OH, OCH₃, OCH₂(CH₂)ₙCH₃, OCH₂(CH₂)ₙCH₂F, OCH₂CH═CH(CH₂)ₙF, O(CH₂)ₙCH═CH(CH₂)ₙI, SH, SCH₃, SCH₂(CH₂)ₙCH₃, SCH₂(CH₂)ₙCH₂F, SCH₂CH═CH(CH₂)ₙF, S(CH₂)ₙCH═CH(CH₂)ₙI (n=0-7), aromatic, haloaromatic, heteroaromatic, haloheteroaromatic, cyclic, and halocyclic, or R₄ and R₅ are taken together with the nitrogen to which they are attached to form a 5- to 7-member heterocyclic ring optionally having O, S or NR₆ in said ring, where R₆ is hydrogen or C₁₋₄ alkyl; X is selected from the group consisting of: X═F, Cl, Br, I, CH₂(CH₂)ₙCH₂F, CH₂CH═CH(CH₂)ₙF, (CH₂)ₙCH═CH(CH₂)ₙI, OH, OCH₃, OCH₂(CH₂)ₙCH₃, OCH₂(CH₂)ₙCH₂F, OCH₂CH═CH(CH₂)ₙF, O(CH₂)ₙCH═CH(CH₂)ₙI, SH, SCH₃, SCH₂(CH₂)ₙCH₃, SCH₂(CH₂)ₙCH₂F, SCH₂CH═CH(CH₂)ₙF, S(CH₂)ₙCH═CH(CH₂)ₙI (n=0-7), and Tc-99m complexed compounds; A, B, C, and D are each independently selected from the group consisting of: C, CH, N, and N⁺O⁻; E and F are each independently selected from the group consisting of: C, CH and N.

21. The compound of claim 20 having one of the formulas:

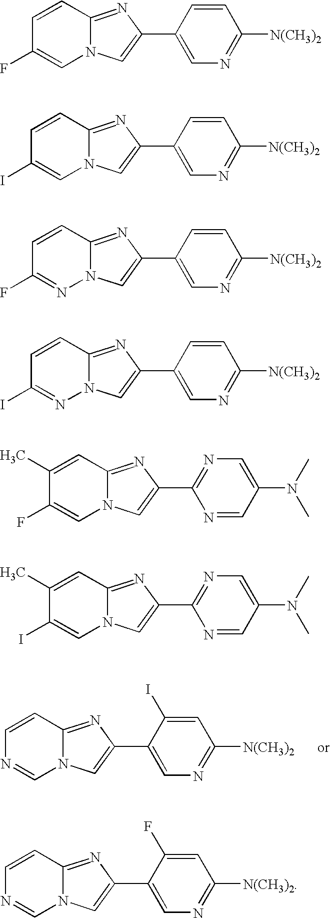

22. (canceled)

23. (canceled)

24. (canceled)

25. (canceled)

26. (canceled)

27. (canceled)

28. (canceled)

29. The compound of claim 20, wherein A, B, and C are C; F is CH; and D and E are N.

30. The compound of claim 1 wherein X is selected from the group consisting of ¹⁸F, ¹²⁵I, ¹³¹I, ¹²³I, ¹²⁴I ⁷⁶Br and ⁷⁷Br.

31. The compound of claim 1 wherein R₃ and R₄ are independently selected from the group consisting of: hydrogen, C₁₋₄ alkyl, C₁₋₄ haloalkyl, and 4-fluorobenzyl.

32. The compound of claim 10 wherein R₄ and R₅ are independently selected from the group consisting of: hydrogen, C₁₋₄ alkyl, C₁₋₄ haloalkyl, and 4-fluorobenzyl.

33. The compound of claim 1 wherein X is ¹²³I, ¹²⁴I or ¹²⁵I and R₃ and R₄ are both methyl.

34. The compound of claim 10 wherein X is ¹²³I, ¹²⁴I or ¹²⁵I and R₄ and R₅ are both methyl.

35. The compound of claim 1 wherein X is F or ¹⁸F, R₁ is methyl and R₂ is H, A is CH, and R₃ and R₄ are methyl.

36. The compound of claim 10 wherein X is F or ¹⁸F, R₂ is methyl and R₃ is H, F is CH, and R₄ and R₅ are methyl.

37. (canceled)

38. (canceled)

39. (canceled)

40. (canceled)

41. A pharmaceutical composition comprising a compound of formula I, II or III, and a pharmaceutically acceptable carrier.

42. A diagnostic composition for imaging amyloid deposits, comprising a radiolabeled compound of claim 41.

43. A method of inhibiting amyloid plaque aggregation in a mammal, comprising administering the composition of claim 41 in an amount effective to inhibit amyloid plaque aggregation.

44. A method of imaging amyloid deposits, comprising: a) introducing into a mammal a detectable quantity of a diagnostic composition of claim 42; b) allowing sufficient time for the labeled compound to become associated with amyloid deposits; and c) detecting the labeled compound associated with one or more amyloid deposits.

45. The compound of claim 10 wherein X is selected from the group consisting of ¹⁸F, ¹²⁵I, ¹³¹I, ¹²³I, ¹²⁴I ⁷⁶Br and ⁷⁷Br.

46. The compound of claim 20 wherein X is selected from the group consisting of ¹⁸F, ¹²⁵I, ¹³¹I, ¹²³I, ¹²⁴I ⁷⁶Br and ⁷⁷Br.

47. The compound of claim 20 wherein R₄ and R₅ are independently selected from the group consisting of: hydrogen, C₁₋₄ alkyl, C₁₋₄ haloalkyl, and 4-fluorobenzyl.

48. The compound of claim 20 wherein X is ¹²³I, ¹²⁴I or ¹²⁵I and R₄ and R₅ are both methyl.

49. The compound of claim 20 wherein X is F or ¹⁸F, R₂ is methyl and R₃ is H, F is CH, and R₄ and R₅ are methyl.
